(19) United States
(12) Patent Application Publication
(10) Pub. No.: US 2010/0210970 A1
(43) Pub. Date: Aug. 19, 2010

(54) PIERCING DEVICE, BLOOD INSPECTION DEVICE, AND PIERCING METHOD

(75) Inventors: Kiyohiro Horikawa, Ehime (JP); Yoshinori Amano, Ehime (JP); Toshiki Matsumoto, Ehime (JP)

Correspondence Address: GREENBLUM & BERNSTEIN, P.L.C., 1950 ROLAND CLARKE PLACE, RESTON, VA, 20191, US

(73) Assignee: PANASONIC CORPORATION, Osaka (JP)

(21) Appl. No.: 12/669,257

(22) PCT Filed: Jul. 18, 2008
(86) PCT No.: PCT/JP2008/001940

§ 371 Date: Jan. 15, 2010

(30) Foreign Application Priority Data
Jul. 18, 2007 (JP) 2007-186641

(51) Int. Cl. A61B 5/00; A61B 17/34

(52) U.S. Cl. 600/583; 606/181

(57) ABSTRACT

A piercing device, a blood inspection device, and a piercing method that facilitate timing management for piercing. The piercing device has a housing (22), a laser emission device (26) being piercing means provided in the housing (22), a holding section (25) provided facing the laser emission device (26), negative pressure means (28) for applying negative pressure to a negative pressure chamber (28a) provided at the holding section (25), an electric circuit section (27) for controlling the negative pressure means (28), a first skin sensor (28b) for causing the laser emission device (26) to prepare for operation, and a timer (27k) for automatically outputting a preparation-for-piercing-is-ready signal based on an output from the first skin sensor (28b). Based on the output from the timer (27k), the laser emission device (26) being the piercing means emits a laser beam (26h) to perform piercing.

### TECHNICAL FIELD

The present invention relates to a puncturing apparatus that punctures skin for example, a blood test apparatus using the puncturing apparatus and a puncturing method.

### BACKGROUND ART

Diabetes patients need to measure their blood sugar level on a regular basis and inject insulin based on the measured blood sugar level to maintain a normal blood sugar level. To maintain this normal blood sugar level, diabetes patients need to measure the blood sugar level on a regular basis. Therefore, patients puncture skin of such as their fingers using a blood test apparatus, sample a small amount of blood exuding from the skin and analyze the component such as blood sugar level, based on the sampled blood.

Conventionally, the blood test apparatuses disclosed in Patent Document 1 and Patent Document 2 have been known. Now, the procedure of a blood test using the blood test apparatus disclosed in Patent Document 1 will be described.

First, the patient touches the blood test apparatus with a finger of one hand (e.g. the index finger of the left hand), pushing a puncturing button of the blood test apparatus by the other hand (e.g. the right hand) and ejecting a puncture needle from a lancet to puncture the skin, so that a droplet of blood is formed on the surface of the skin. Next, the patient brings one of blood sensors stacked and stored in a cartridge installed in the blood test apparatus close to the puncturing position to make the sensor touch the blood. By this means, the blood test apparatus analyzes the components of the blood taken into the blood sensor.

Now, a conventional puncturing apparatus will be described below as an example, using a blood test apparatus.

FIG. 1 is a cross sectional view showing a configuration of the blood test apparatus described in Patent Document 1.

In FIG. 1, blood test apparatus 1 has: a substantially rectangular solid shaped housing; cartridge 3, blood sensors 4 stacked and stored in cartridge 3; blood sensor outlet 3a provided in cartridge 3; puncturing section 5 to which blood sensor 4a conveyed from blood sensor outlet 3a is mounted; needle puncture section 6 that is provided facing the puncturing section 5 and punctures skin 9 with puncture needle 6a; electrical circuit section 7 electrically connected to blood sensor 4a conveyed to puncturing section 5; and conveying means 8 that conveys the blood sensors 4 stored in cartridge 3 to puncturing section 5 one-by-one. Here, cartridge 3, blood sensors 4, blood sensor outlet 3a, puncturing section 5, needle puncture section 6, electrical circuit section 7 and conveying means 8 are housed in housing 2.

In addition, puncturing button 6b that shoots puncturing needle 6a and conveying button 8a that activates conveying means 8 are provided on a surface of housing 2. Here, recently there is a blood test apparatus provided with a negative pressure means (not shown in the figure), and the negative pressure means applies a negative pressure to the vicinity of puncturing section 5 to facilitate blood 10 exuding.

FIG. 2 is a flowchart showing a test method using blood test apparatus 1. FIG. 3 is a drawing explaining a usage condition of blood test apparatus 1.

First, in step S1, blood test apparatus 1 is held by the right hand (see FIG. 3), while the skin 9 of the index finger of the left hand touches puncturing section 5.

In step S2, puncturing button 6 is depressed by the thumb of the right hand. The step moves to step S3 by the depressing of puncturing button 6b.

In step S3, skin 9 of the index finger of the left hand is punctured. The blood exudes from punctured skin 9.

In step S4, it is checked that the blood has sufficiently exuded. The user waits until blood 10 has sufficiently exuded and depresses conveying button 8a by the middle finger of the right hand while a positional relationship between puncturing section 5 and the left hand is kept constant. The step moves to step S5 when conveying button 8a is depressed.

In step S5, conveying means 8 is activated to convey blood sensor 4a to puncturing section 5. The blood 10 exuding from the skin 9 is then taken into blood sensor 4a.

In step S6, electrical circuit section 7 measures the property of blood 10 taken into the blood sensor 4a, and the flow is terminated.

After depressing puncturing button 6b, the user depresses conveying button 8a using any finger while keeping the state of blood test apparatus 1 so as not to move from the skin 9. The reason for keeping the state so as not to move blood test apparatus 1 from the skin is to make a part of blood sensor 4a (blood guiding part) attach to exuded blood 10. If the finger (skin 9) is moved, a large amount of blood 10 is required to attach blood sensor 4a to blood 10, the reliability is reduced and a heavy burden is imposed on the patient.

Here, for the blood test apparatus that assists to exude blood using a negative pressure means, it is necessary to control a pressing timing of the negative pressure button for activating the negative pressure means and also a pressing timing of puncturing button 6b.
Paten Document 1: Japanese Patent Application Laid-Open No. 2004-519302
Patent Document 2: Japanese Patent Application Publication No. 2003-339680

### DISCLOSURE OF INVENTION

### Problems to be Resolved by the Invention

However, in order to perform a blood test using such conventional blood test method, the patient has to observe a state where blood 10 is exuding while holding blood test apparatus 1 and depressing puncturing button 6b. Then, the patient has to depress conveying button 8a while fixing blood test apparatus 1 at the timing at which the patient judges that an appropriate amount of blood has exuded.

Therefore, there has been a problem that it is greatly difficult to adjust the timing for depressing the button and it lacks certitude. Moreover, when the puncturing apparatus having the negative pressure means is employed, the timing control becomes further complicated because an appropriate timing to depress a negative pressure button has to be also controlled.

It is therefore an object of the present invention is to provide a puncturing apparatus, a blood test apparatus and a puncturing method that can solve the above-described problem and easily control the timing to puncture.

### Means for Solving the Problem

A puncturing apparatus according to the present invention includes: a housing; a puncturing section that is provided in the housing; a first skin detecting section that detects that skin touches a predetermined puncturing position; a second skin detecting section that detects swelled skin after the first detecting section detects contact with the skin; a control section that allows the puncturing section to puncture the swelled skin when the second skin detecting section detects the swelled skin.

A blood test apparatus according to the present invention includes a puncturing section that punctures skin though a blood sensor to test blood exuding on the blood sensor by puncturing. The above-described puncturing apparatus is used as the puncturing section.

A puncturing method according to the present invention includes: detecting that skin touches a predetermined puncturing position of a housing; detecting swelled skin after detecting contact with the skin; and puncturing the swelled skin when detecting the swelled skin.

A puncturing method according to the present invention includes: detecting that skin touches a predetermined puncturing position of a housing; applying a negative pressure to inside the housing to swell the skin after detecting contact with the skin; detecting swelled skin; puncturing the swelled skin when detecting the swelled skin.

A puncturing method according to the present invention includes: detecting that skin touches a predetermined puncturing position of a housing; applying a negative pressure to inside the housing to swell the skin after detecting contact with the skin; starting charging a capacitor for emitting laser light; detecting that charging on the capacitor is completed; detecting swelled skin; and puncturing the swelled skin with the laser light when detecting the swelled skin and detecting that the charging is completed.

### Advantageous Effects of Invention

According to the present invention, puncturing can be automatically performed at a timing suitable for puncturing and can be performed reliably. In addition, since puncturing can be automatically performed, the operation becomes significantly easier. Particularly, even if the puncturing apparatus has a negative pressure means, puncturing can be automatically performed at a timing suitable for puncturing. Consequently, an effect of allowing an unskilled patient to surely puncture to complete a blood test by puncturing once can be expected.

### BRIEF DESCRIPTION OF DRAWINGS

FIG. 1 is a cross sectional view of a conventional blood test apparatus;

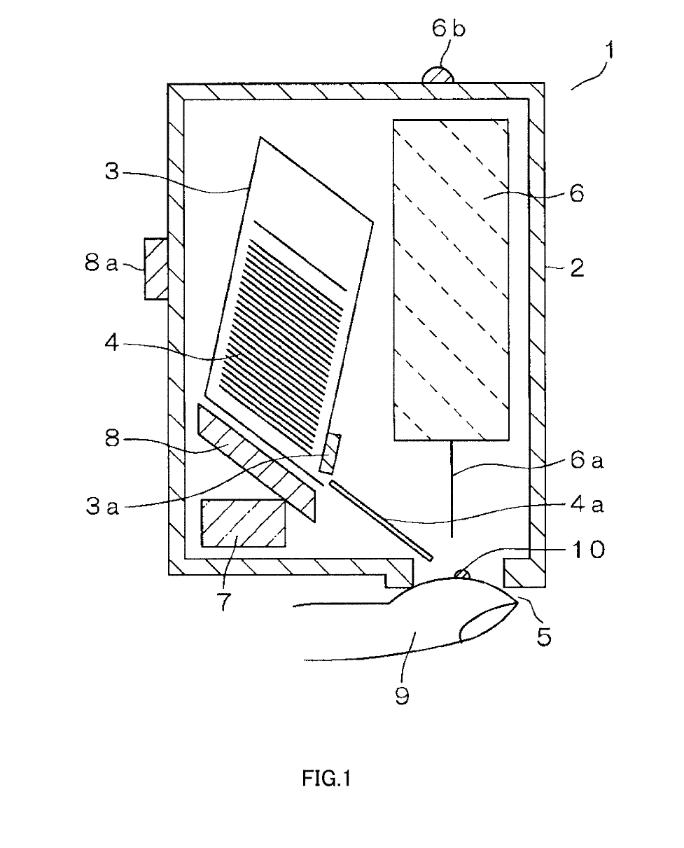

FIG. 2 is a flowchart of a conventional blood test apparatus;

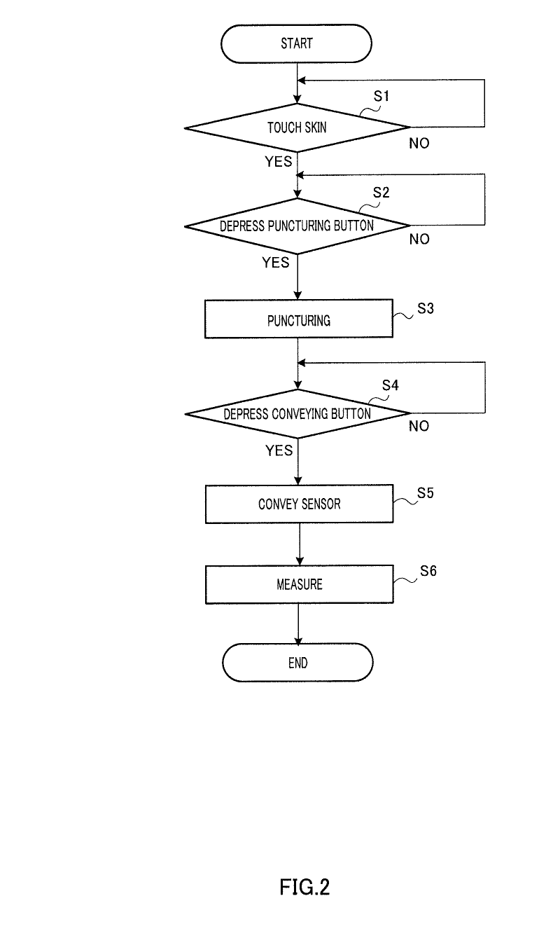

FIG. 3 is an explanation drawing of a conventional blood test apparatus in use;

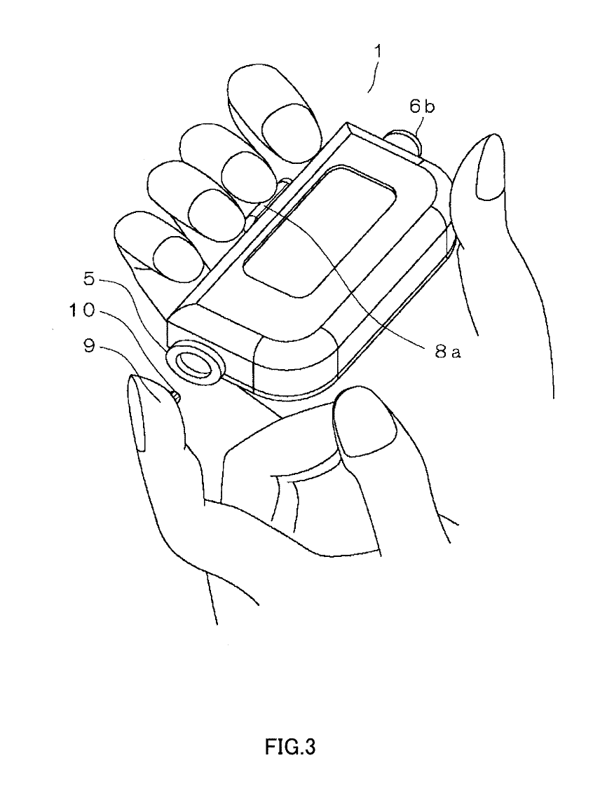

FIG. 4 is a perspective plan view showing a configuration of a cartridge of a blood test apparatus according to embodiment 1 of the present invention;

FIG. 5 is a cross sectional view of blood sensors stacked and stored in the blood test apparatus according to embodiment 1;

FIG. 6 is a perspective plan view of a blood sensor of the blood test apparatus according to embodiment 1;

FIG. 7 is an external perspective view of a blood sensor of the blood test apparatus according to embodiment 1;

FIG. 8 is a perspective plan view of a cartridge of a blood sensor of the blood test apparatus according to the embodiment 1;

FIG. 9 is a cross sectional view of a laser emitting device being an example of puncturing means of the blood test apparatus according to embodiment 1;

FIG. 10 is a side view of a first holder constituting a holding section of the blood test apparatus according to embodiment 1;

FIG. 11 is an external perspective view of the first holder constituting the holding section of the blood test apparatus according to embodiment 1;

FIG. 12 is a cross sectional side view of a second holder constituting the holding section of the blood test apparatus according to embodiment 1;

FIG. 13 is a cross sectional view of the holding section in puncturing-and-measuring using the blood test apparatus and its nearby primary parts according to embodiment 1;

FIG. 14 is a block diagram of an electrical circuit section and its periphery constituted by only the puncturing apparatus of the blood test apparatus according to embodiment 1;

FIG. 15 is a block diagram of the electrical circuit section and its periphery of the blood test apparatus according to embodiment 1;

FIG. 16 is a flowchart showing a puncturing method for the puncturing apparatus of the blood test apparatus according to embodiment 1;

FIG. 17 is a flowchart showing a test method for the blood test apparatus according to embodiment 1;

FIG. 18 is a drawing explaining a reference position of the puncture depth for a puncturing apparatus according to embodiment 2 of the present invention;

FIG. 19 is a drawing explaining a reference position of the puncture depth for the puncturing apparatus according to embodiment 2;

FIG. 20 is a drawing explaining a method of detecting swelled skin in the puncturing apparatus according to embodiment 2;

FIG. 21 is a drawing explaining a method of detecting swelled skin in the puncturing apparatus according to embodiment 2;

FIG. 22 is a drawing explaining a method of detecting swelled skin in the puncturing apparatus according to embodiment 2;

FIG. 23 is a drawing explaining a method of detecting swelled skin in the puncturing apparatus according to the embodiment 2;

FIG. 24 is a drawing explaining a method of detecting swelled skin for the puncturing apparatus according to embodiment 2;

FIG. 25 is a flowchart showing a puncturing-and-measuring method for the puncturing apparatus according to embodiment 2;

FIG. 26 is a flowchart showing a puncturing-and-measuring method when a failure occurs because preparation for puncturing of the puncturing apparatus is not completed according to embodiment 3 of the present invention; and

FIG. 27 is a flowchart showing a puncturing-and-measuring method when a failure occurs because puncturing-and-measuring by the puncturing apparatus is not successful according to embodiment 3.

### BEST MODE FOR CARRYING OUT THE INVENTION

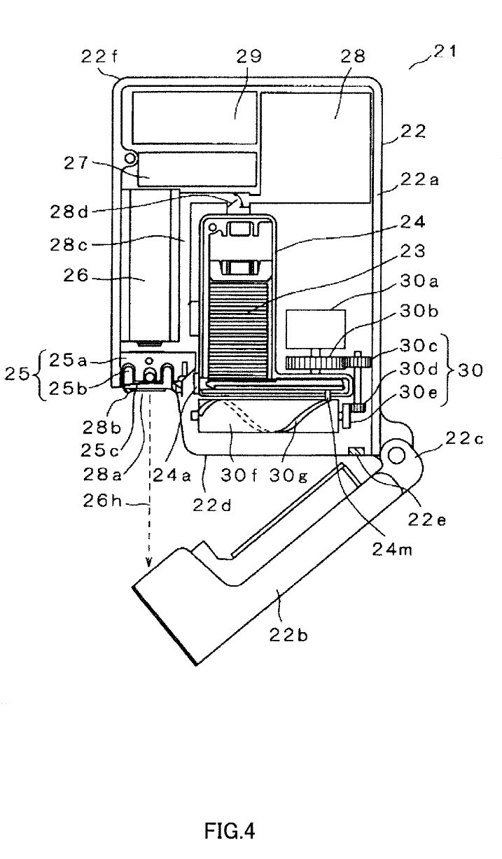

Now embodiments of the present invention will be explained based on the accompanying drawings. Here, in description of each embodiment, directions such as top and bottom are defined, on the basis of the blood test apparatus in use. The upward direction of FIG. 4 means the upward direction of the blood test apparatus in use.

### Embodiment 1

FIG. 4 is a perspective plan view showing a configuration of a blood test apparatus according to embodiment 1 of the present invention. The present embodiment is suitable for a blood test apparatus having a puncturing apparatus that punctures skin through a blood sensor.

In FIG. 4, a blood test apparatus 21 has housing 22, cartridge 24, holding section 25, laser emitting device 26, electrical circuit section 27, negative pressure means 28, battery 29 and conveying means 30, and here cartridge 24, holding section 25, laser emitting device 26, electrical circuit section 27, negative pressure means 28, battery 29 and conveying means 30 are housed in housing 22.

Housing 22 has main body 22a and cover 22b. Cover 22b is pivotably coupled to main body 22a with a centrally-located supporting point 22c. Rotation of cover 22b stops in a state where n housing 22 is closed, that is, rotation of cover 22b stops in a state where the opposite end of supporting point 22c is in contact with main body 22a. In addition, cover 22b comes to rest in the first resting position where cover 22b is open about 30 degrees from main body 22a, and the second resting position where cover 22b is open about 90 degrees from main body 22a. Opening and closing of cover 22b is detected by cover detecting sensor 22e provided on one side 22d of main body 22a.

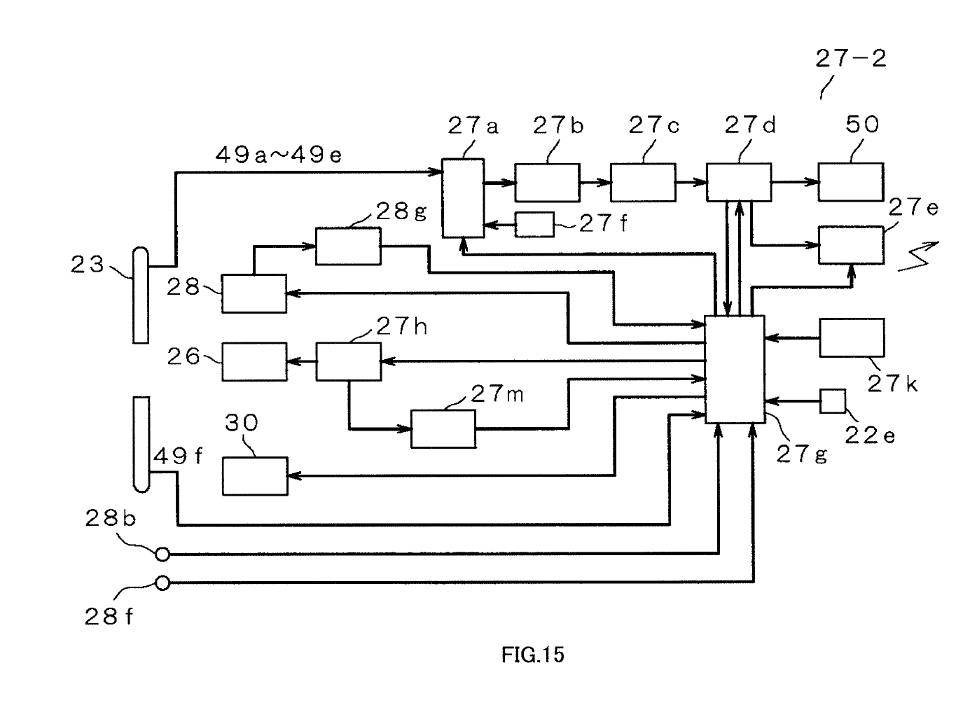

Laser emitting device 26, which is an example of puncturing apparatus prepares puncturing by taking a trigger that the skin of the patient touches first skin detecting sensor 28b of negative pressure chamber 28a (described later), and when various preparations for puncturing (described later) are completed, an electrical signal indicating that each preparation for puncturing is completed is input to control section 27g (see FIG. 15) in electric circuit section 27.

Cartridge 24 is removably mounted on main body 22a, and stacks and stores therein the sensors 23. Cover 22b is rested at the second resting position, so that cartridge 24 can be easily replaced. Here, the internal configuration of the cartridge 24 will be described in detail later.

Holding section 25 is composed of first holder 25a provided above, and second holder 25b that faces first holder 25a and is provided below. First holder 25a is fixed to the side surface of main body 22a and second holder 25b is biased toward first holder 25 by spring 25c. One blood sensor 23 conveyed from sensor outlet 24a of cartridge 24 is sandwiched and fixed between first holder 25a and second holder 25b.

First holder 25a is fixed to main body 22a and also laser emitting device 26 is fixed to main body 22a, so that the distance from laser emitting device 26 to second holder 25b is kept constant. Consequently, puncturing can be performed with the puncture depth according to a set value. Here, this result can be obtained by biasing first holder 25a by the spring while second holder 25b is fixed.

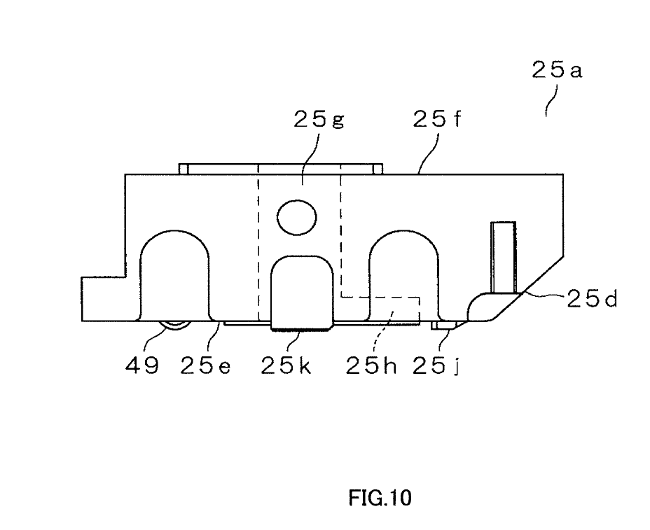

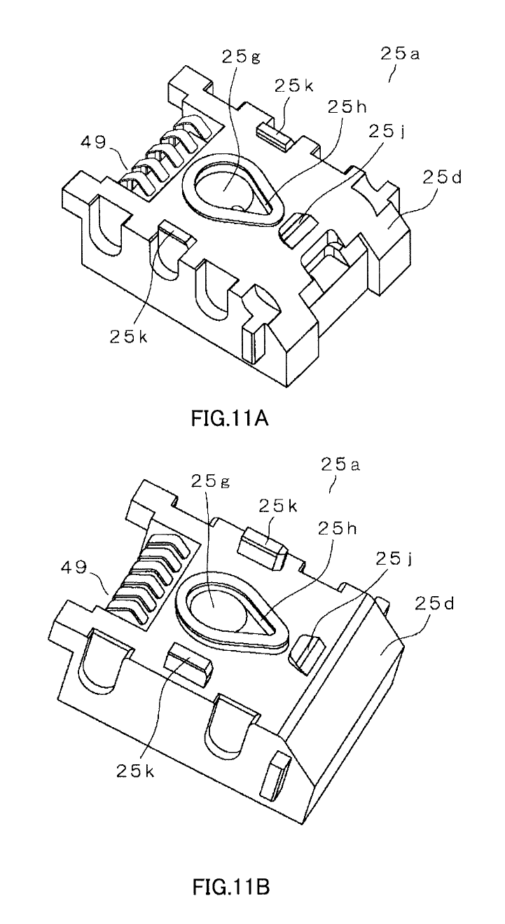

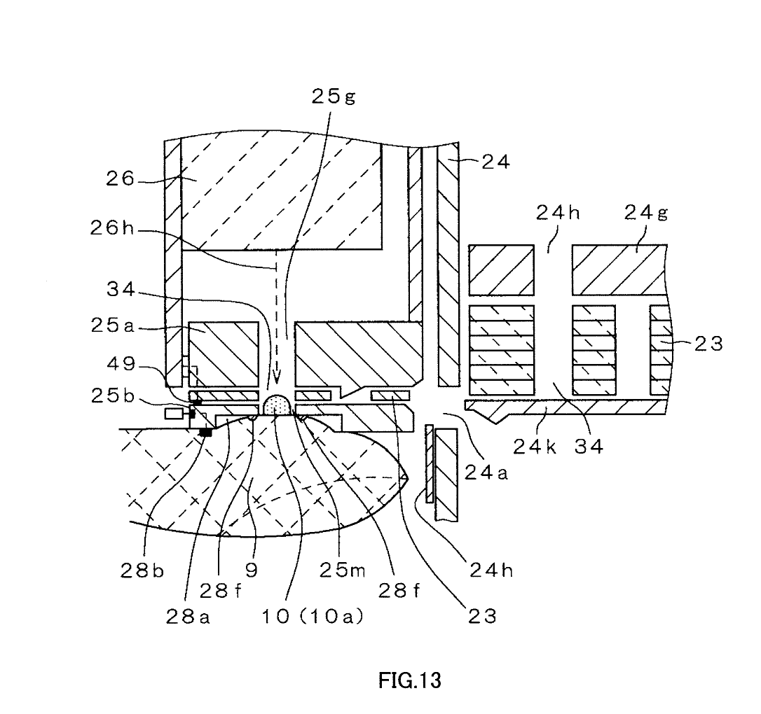

Negative pressure chamber 28a is formed below holding section 25 connected to negative pressure means 28 through negative pressure means 28. Moreover, first detecting sensor 28b (an example of preparation for puncturing) that detects skin 9 (see FIG. 13 described later) is provided, adjacent to negative pressure chamber 28a, on the under surface of second holder 25b constituting holding section 25. Holding section 25 will be described in detail with reference to FIG. 10 and FIG. 11.

Laser emitting device 26, which is a puncturing means, is fixed in a position in the back of holding section 25 (the upward position in FIG. 4). Laser emitting device 26 emits laser light 26h to puncture the skin of the patient in contact with holding section 25. When laser light 26h is emitted, cover 22b is rested at the first resting position at the opening angle about 30 degrees, so that laser light 26h hits against a part of cover 22b and does not leak outside, thereby ensuring security.

Here, although an instance where laser emitting device 26 is used as a puncturing means is described in the preset embodiment, the present invention is not limited to this, and a needle puncturing apparatus with a puncture needle may be used.

Electrical circuit section 27 is electrically connected with blood sensors 23, first skin detecting sensor 28b, second skin detecting sensor 28f, laser emitting device 26 and negative pressure means 28. Then, when being ready for puncturing, electrical circuit 27 allows laser emitting device 26 to emit laser light 26h. In addition, electrical circuit section 27 analyzes the components of blood sampled by blood sensor 23. Moreover, electrical circuit 27 allows negative pressure means 28 to generate a negative pressure at a predetermined timing.

Negative pressure means 28 applies a negative pressure to the vicinity of holding section 25 and the inside of cartridge 24.

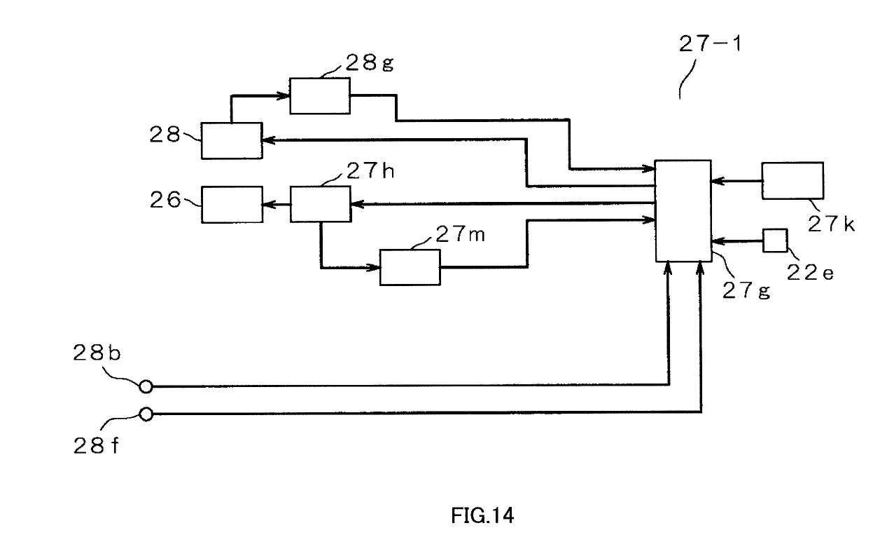

The negative pressure produced by negative pressure means 28 is guided into negative pressure chamber 28a of holding section 25 through negative pressure path 28c. In addition, the negative pressure is also guided into cartridge 24 through negative pressure path 24f. Switching valve 28d switches between negative pressure path 28c and negative pressure path 24f to guide the negative pressure. Control section 27g in electrical circuit section 27 controls switching valve 28d and controls to turn electrical circuit 27 on and off. Negative pressure means 28 has current change detecting section 28g that detects change in a current (see FIG. 14 and FIG. 15 described later).

Battery 29 supplies electric power to laser emitting device 26, electrical circuit section 27 and negative pressure means 28.

Conveying means 30 separates one blood sensor from blood sensors 23 stacked and stored in cartridge 24 and conveys the one blood sensor to holding section 25.

Conveying means 30 is composed of motor 30a connected to electrical circuit section 27 and cylindrical body 30f connected to motor 30a through gears 30b, 30c, 30d and 30e. Spiral groove 30g is formed on the surface of cylindrical body 30f. Projection 24m formed in slider 24k is slidably inserted in spiral groove 30g.

Accordingly, the rotation of motor 30a allows cylindrical body 30f to rotate through gears 30b, 30c, 30d and 30e. Rotation of cylindrical body 30f causes projection 24m inserted in groove 30 to linearly move on cylindrical body 30f. That is, slider 24k moves, thereby the blood sensor 23 is conveyed from sensor outlet 24a and is mounted to the holding section 25.

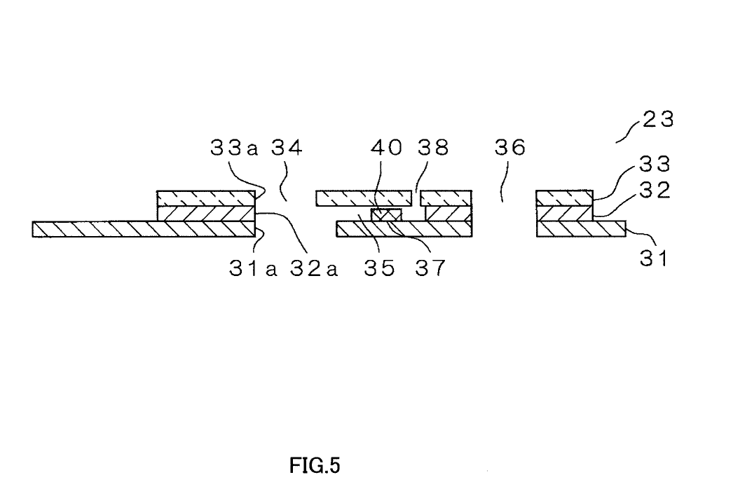

FIG. 5 is a cross sectional view of blood sensors 23 stacked and stored in cartridge 24.

In FIG. 5, blood sensor 23 has a plate-like body, and is composed of substrate 31, spacer 32 pasted on the upper surface of substrate 31, and cover 33 pasted on the upper surface of spacer 32.

Sensor 23 has opening 34 for storing blood. Opening 34 is provided on a position through which laser light 26h passes when sensor 23 is mounted in holding section 25.

Opening 34 is an opening composed of substrate hole 31a formed on substrate 31, spacer hole 32a formed on spacer 32 and cover hole 33a formed on cover 33.

Supply path 35 is a guide path that is coupled to storing section 34 at one end thereof, and guides blood 10 stored in storing hole 34 to detecting section 37 by utilizing capillary action. The other end of this supply path 35 is coupled to air hole 38. Here, the capacity of opening 34 is about 1 μL, and the capacity of supply path 35 is about 0.15 μL. Thus, it is possible to perform a test using a small amount of blood 11, so that the burden on the patient can be reduced.

Positioning hole 36 penetrates blood sensor 23 and determines the mounting position of blood sensor 23.

Detecting section 37 measures the blood sugar level and so forth of blood 10.

Reagent 40 is arranged on detecting section 37. This reagent 40 can be obtained by adding and dissolving PQQ-GDH (0.1 to 5.0 U/sensor), potassium ferricyanide (10 to 200 millimole), maltitol (1 to 50 millimole) and taurine (20 to 200 millimole) in a CMC solution of 0.01 to 2.0 wt % to prepare a reagent solution, by dropping the reagent solution on detecting section 37 formed on substrate 31 and drying. This reagent 40 is progressively degraded due to moisture absorbent.

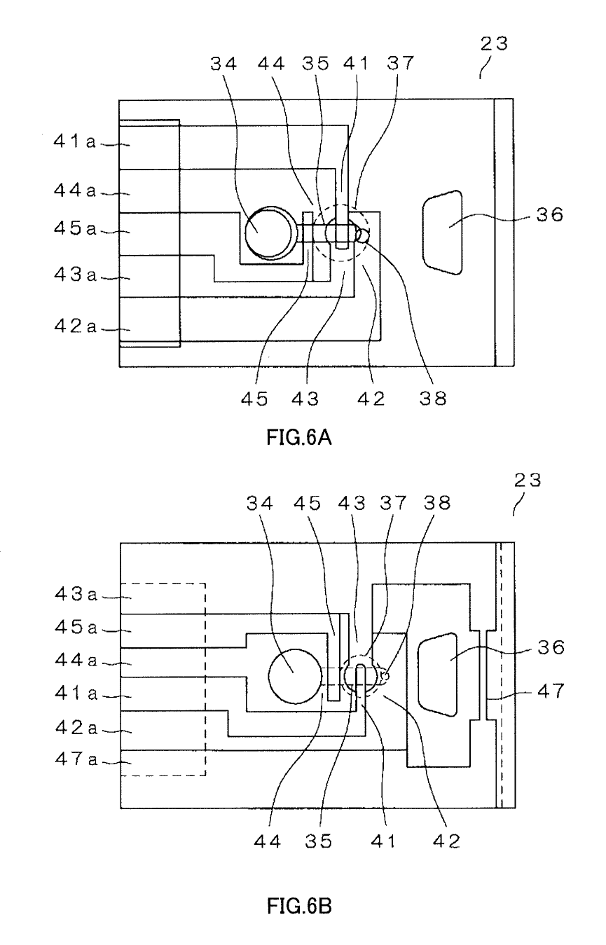

Here, an electrically conductive layer is formed on the upper surface of the substrate 31 by the sputtering method or the vapor deposition method using material such as gold, platinum, or palladium. Detection electrodes 41 to 45 (see FIG. 6(A) and FIG. 6(B)), connection electrodes 41a to 45a derived from these detection electrodes 41 to 45 and an identification electrode 47a (as shown in FIG. 6(B)) are integrally formed by applying laser machining to the electrically conductive layer. Polyethylene terephthalate (PET) is used for the material of substrate 31, spacer 32 and cover 33. The material is used common to these components in this way, so that the management cost can be reduced.

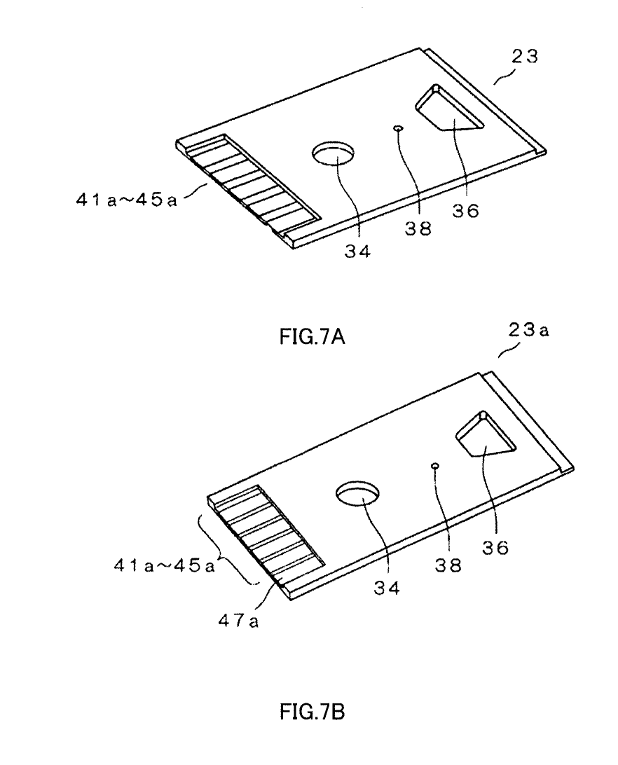

FIG. 6 is a perspective plan view of blood sensor 23, and FIG. 7 is an external perspective view of blood sensor 23. FIG. 6(A) and FIG. 7(A) show sensor 23 having 5 electrodes not including identification electrode 47a, and FIG. 6(B) and FIG. 7(B) show sensor 23 having 6 electrodes including identification electrode 47a.

Now, sensor 23 will be described with reference to FIG. 6(B) and 7(B) with 6 electrodes.

Opening 34 is formed approximately at the center of plate-like sensor 23, connection electrodes 41a to 45a and identification electrode 47a are formed at one end of sensor 23, and positioning section 36 is formed at the other end of sensor 23. Positioning section 36 is a hole, and has a trapezoidal shape narrowing toward opening 34. Air hole 38 is formed between positioning section 36 and opening 34.

Supply path 35 having one end connected to opening 34 faces detection electrode 42. Meanwhile, the other end of supply path 35 is coupled to air hole 38.

Opening 34, detection electrode 44 connected to connection electrode 44a, detection electrode 45 connected to connection electrode 45a, again detection electrode 44 connected to connection electrode 44a, detection electrode 43 connected to connection electrode 43a, detection electrode 41 connected to connection electrode 41a, again detection electrode 43 connected to connection electrode 43a and detection electrode 42 connected to connection electrode 42a, are provided on supply path 35, in the order described. In addition, reagent 40 (see FIG. 5) is placed on detection electrodes 41 and 43. Identifying section 47 formed by a conductor pattern is formed between detection electrode 43 and identification electrode 47a.

Blood test apparatus 21 (see FIG. 5) can detect whether there is electrical conduction between connection electrode 43a and identification electrode 47a to identify whether sensor 23 is mounted in holding section 25. That is, in case where there is not electrical conduction when conveying means 30 conveys sensor 23 to holding section 25, blood test apparatus 21 can display on display section 50 (see FIG. 15) a warning indicating that sensor 23 is not mounted in holding section 25.

It is possible to store information of the calibration curve and also manufacturing information by changing the electric resistance of identifying section 47. Therefore, a blood test can be more accurately performed by using those information.

Here, FIG. 6(A) shows an instance where blood sensor 23 has 5 electrodes, (i.e. the number of electrode of FIG. 6(A) is one less than that of FIG. 6(B) and also shows an instance where there is not identification electrode 47a. It is possible to assign the identification electrode to one electrode which is, for example, a detection electrode other than a working electrode and a counter electrode (described later) that is not used to measure the components of blood, so that the electrodes can be configured by 5 electrodes. The instance of FIG. 6(A) is the same as that of FIG. 6(B) other than the number of electrodes, i.e. FIG. 6(A) has 5 electrodes but FIG. 6(B) has 6 electrodes. Even if the number of electrodes is five, automatic identification can be performed by the electrodes as with FIG. 6(B) having 6 electrodes.

In addition, although sensor 23 shown in FIG. 6 and FIG. 7 is formed of a rectangular plate-like body, the shape of sensor 23 is not limited, and here, the shape of sensor 23 may be a square and a polygon other than a quadrangle, or a semicircle and so forth.

Moreover, the shape of positioning section 36 is not limited, and here, positioning section 36 may be a hole shaped as a quadrangle, a polygon other than a quadrangle, a semicircle, a circle or an oval. Furthermore, positioning section 36 may not be a hole but may be a concave part although not shown in the figure.

Next, the internal structure of cartridge 24 will be described in detail.

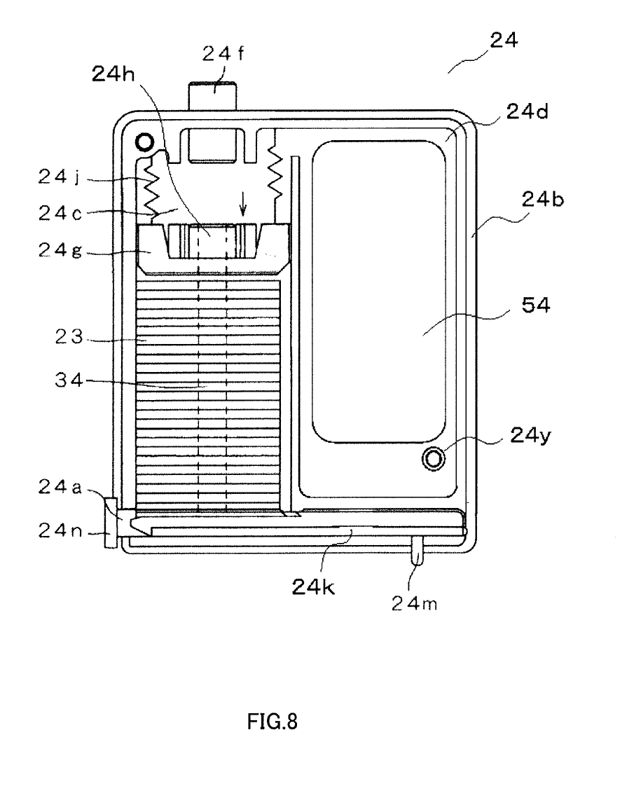

FIG. 8 is a perspective plan view of cartridge 24.

In FIG. 8, case 24b is made of resin and includes sensor chamber 24c and drying chamber 24d. Blood sensors 23 are stacked and stored in sensor chamber 24c. Desiccant 54 is stored in drying chamber 24d.

Negative pressure path 24f formed on the upper part of sensor chamber 24c is connected to negative pressure means 28 (not shown in the figure) and supplies a negative pressure into sensor chamber 24c to reduce the dampness in sensor chamber 24c. Spring 24j biases stacked and stored blood sensors 23 downward through pressure plate 24g. At approximately the center of pressure plate 24g, hole 24h is provided on a position in communication with opening 34 of blood sensor 23.

Consequently, the negative pressure guided from negative pressure path 24f reduces the dampness in opening 34 of each blood sensor 23 through hole 24h, and protects reagent 40 (see FIG. 5) installed in each sensor 23 from deteriorating due to the dampness.

In addition, pressure sensor 24y that measures the degree of a negative pressure in drying chamber 24d is mounted in drying chamber 24d. Based on the measurement result by pressure sensor 24y, the negative pressure in the sensor chamber 24c and the drying chamber 24d can be appropriately controlled through negative pressure outlet 24f such that the negative pressure is not more than a predetermined value, so that blood sensors 23 can be always dry.

Slider 24k sliding below sensor chamber 24c is a part forming conveying means 30. This slider 24k is provided with a projection 24m that is moved toward sensor outlet 24a by conveying means 30.

Conveying means 30 composed of slider 24k, projection 24m and spring (not shown in the figure) is formed below sensor chamber 24c. Slider 24k has a flat plate shape and is securely clamped to projection 24m. Slider 24k separates the undermost blood sensor 23 from the blood sensors stacked and stored in cartridge 24 and conveys this blood sensor 23 from sensor outlet 24a to the outside.

Although an instance where the conveying operation is electrically performed using a motor has been described above, the conveying operation may be performed manually by the patient.

The patient moves projection 24m to slide slider 24k toward sensor outlet 24a along a groove provided on the lower part of case 24b. The undermost blood sensor 23 can be separated and conveyed toward holding section 25 (see FIG. 4) by sliding slider 24k toward sensor outlet 24a. In addition, when the patient releases his/her hand from projection 24m, slider 24k automatically returns to the inside of case 24b because of being biased by a spring (not shown in the figure). Accordingly, battery 29 is not consumed.

Shutter 24n opens and closes sensor outlet 24a. Shutter 24n opens in conjunction with opening cover 22b of housing 22 (see FIG. 4). Then, the patient slides projection 24m while shutter 24n opens, so that blood sensor 23 placed on slider 24k can be conveyed from sensor outlet 24a to holding section 25 (see FIG. 4).

In addition, shutter 24n closes in conjunction with closing cover 22b. Sensor outlet 24a is closed by shutter 24n to seal up the inside of sensor 24c, so that the negative pressure is held.

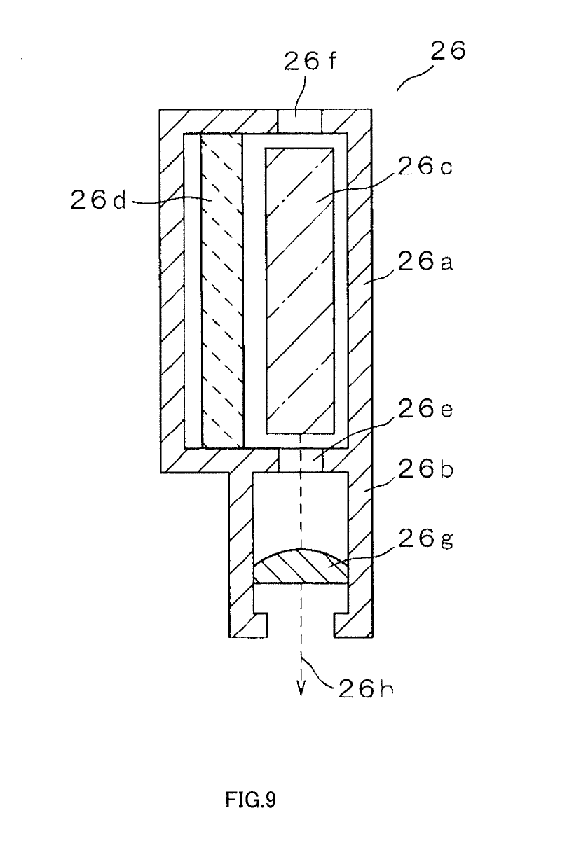

FIG. 9 is a cross sectional view of laser emitting device 26, which is a puncturing means.

In FIG. 9, laser emitting device 26 is composed of oscillating tube 26a and tubular body 26b coupled to this oscillating tube 26a. Er:YAG (yttrium, aluminium, garnet) laser crystal 26c and excitation light source 26d are housed in oscillating tube 26a.

Excitation light source 26d is connected to high voltage generating circuit 27h provided in electrical circuit section 27. This high voltage generating circuit 27h is provided with charge completion detecting section 27m that detects the completion of charging.

Partially transmitting mirror 26e having a transmissivity of 3% to 15% is mounted on one end of oscillating tube 26a, and total reflecting mirror 26f having a transmissivity equal to or less than 0.5% is mounted on the other end of oscillating tube 26a. Condenser lens 26g is mounted in tubular body 26b before partially transmitting mirror 26e. By such arrangement, laser light 26h can focus on a position near the surface of skin 9 of the patient.

The operation of laser emitting device 26 having the above-described configuration will be described. When first skin detecting sensor 28b (see FIG. 4) detects skin 9, high voltage generating circuit 27h (see FIG. 14 and FIG. 15 described later) supplies energy charged in a capacitor to excitation light source 26d to emit exciting light at one of: the time at which timer 27k (see FIG. 14 described later) measures a predetermined passage of time (first example of preparation for puncturing is completed); and the time at which current change detecting section 28g detects change in the current supplied to negative pressure means 28 (second example of preparation for puncturing is completed), and also the time at which charge completion detecting section 27m (described later) detects that the capacitor is sufficiently charged via high voltage generating circuit 27h (third example of ready for puncturing).

This exciting light emitted from excitation light source 26d enters Er:YAG laser crystal 26c and excites doped laser operating substance (erbium Er in the present embodiment) to generate light. The generated light is then continuously reflected by total reflecting mirror 26f, YAG laser crystal 26c and partially transmitting mirror 26e, passing through them and resonates, and is amplified. A part of the amplified laser light passes through partially transmitting mirror 26e Laser light 26h passed through partially transmitting mirror 26e passes through focusing lens 26g, is emitted from laser emitting device 26 and focuses on a position near the surface of skin 9. The puncture depth is preferably 0.6 mm to 1.5 mm from the surface of skin 9, here, the depth is 0.8 mm in the present embodiment.

Since the present embodiment employs laser puncturing device 26 being capable of puncturing skin 10 of the patient out of touch, it has a lower risk than a needle puncture means which requires discarding and replacing used needles, and is very sanitary. Further, laser emitting device 26 has no moving components, so that there is little failure.

Next, a configuration of holding section 25 will be described in detail.

FIG. 10 is a side view of first holder 25a constituting holding section 25, and FIG. 11 is an external perspective view of first holder 25a from the bottom surface 25e side.

FIG. 11(A) shows an instance where 5 electrodes are used and FIG. 11(B) shows an instance where 6 electrodes are used. FIG. 11(A) is the same as FIG. 11(B) other than the number of connectors 49. Hereinafter first holder a will be described with reference to FIG. 10 and FIG. 11(B), where 6 electrodes are used.

Hole 25g penetrating from upper surface 25f to under surface 25e is provided on first holder 25a at a position through which laser light 26h passes. Here, when a needle puncturing apparatus is used instead of laser emitting device 26, a puncture needle passes through hole 25g.

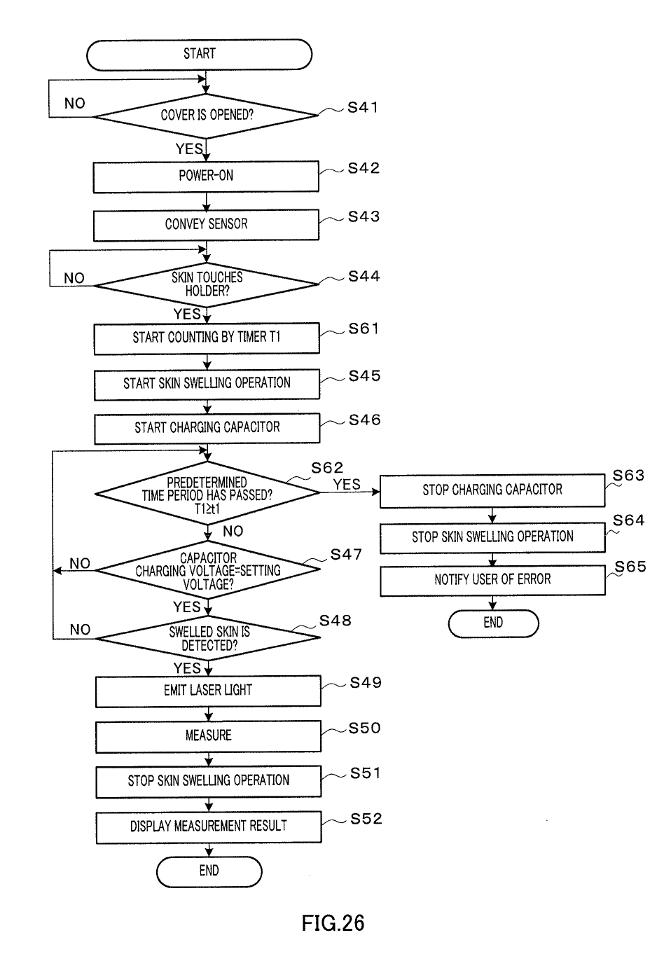

In addition, when blood sensor 23 is mounted in holding section 25, the 25g corresponds to opening 34 (see FIG. 26, 4) provided on the blood sensor 23. The lower part of hole 25g is bent in an L-shape. This L-shaped tip 25h corresponds to air hole 38 formed on blood sensor 23 in order to assure the effect of capillary action of the supply path formed in blood sensor 23. In addition, a negative pressure is supplied through hole 25g.

In addition, notch part 25h is provided on the lower part of hole 25g in order to assure the effect of capillary action through supply path 35 formed on sensor 23. Negative pressure is supplied through hole 25. Here, hole 25m in communication with hole 25g is provided on second holder 25b.

Projection 25j is provided between opening 25d and hole 25g. Projection 25j is engaged with positioning section 36 provided in blood sensor 23 to position the blood sensor 23 in the horizontal direction. Projection 25j has a trapezoidal shape narrowing toward hole 25g. The thickness of projection 25j gradually increases from opening 25d toward hole 25g. Consequently, inserted in holding section 25, sensor 23 is easily fixed in holding section 25.

Convex parts 25k are provided on both sides of first holder 25a, just beside hole 25g. Convex parts 25k position blood sensor 23 in the width direction. Connectors 49 are provided on the opposite side of opening 25d. Connectors 49 are in contact with connection electrodes 41a to 45a and identification electrode 47a, and is connected to electrical circuit section 27. Here, projection 25j and convex parts 25k may be provided on second holder 25b.

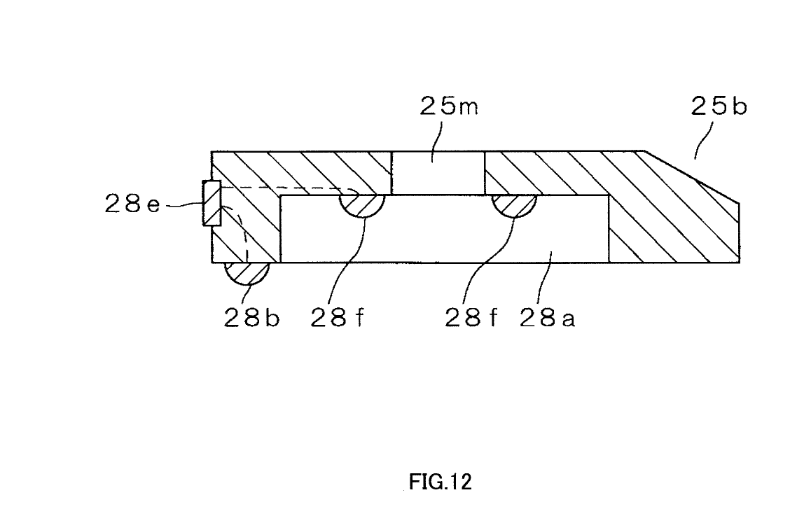

FIG. 12 is a cross sectional side view of second holder 25b constituting holding section 25.

As shown in FIG. 12, hole 25m in communication with hole 25g formed on first holder 25a is provided on second holder 25b. In addition, negative pressure chamber 28a having an opening bottom is coupled with hole 25m. Moreover, first skin detecting sensor 28b is provided adjacent to negative pressure chamber 28a. First skin detecting sensor 28b is connected to electrical circuit section 27 through connector 28e.

Moreover, second skin detecting sensor 28f that detects the swelled skin when a negative pressure is created in negative pressure chamber 28a is provided on the upper surface of negative pressure chamber 28a. Second skin detecting sensor 28f is connected to electrical circuits 27 through connector 28e as with first skin detecting sensor 28b. In this case, connector 28e has one or more connection terminals.

The above-described first detecting sensor 28b, second skin detecting sensor 28f and cover detecting sensor 22e (see FIG. 4) may be mechanical switches or have a function to detect electrical conduction. Moreover, these sensors may be optical sensors employing light emitting elements and light receiving elements, or may be magnetic sensors. In the present embodiment, electrical sensors that detect the electric resistance of skin 9 are used as first skin detecting sensor 28b and second skin detecting sensor 28f, and a mechanical switch is used as cover detecting sensor 22e.

Here, although skin 9 swells by applying a negative pressure to negative pressure chamber 28a, the present embodiment is limited to this, and skin 9 may be strongly pressed against second holder 25b. In addition, although “negative pressure chamber 28a” is employed as a component name for convenience of explanation, negative pressure may not always be used.

FIG. 13 is a cross sectional view of the holding section in puncturing-and-measuring using blood test apparatus 21 and its nearby primary parts.

As shown in FIG. 13, negative pressure chamber 28a is formed on the under surface of second holder 25b. Negative pressure chamber 28a is connected to negative pressure means 28 (see FIG. 4) through hole 25m formed on second holder 25b, opening 34 of blood sensor 23 and hole 25g of first holder 25a. First skin detecting chamber 28b is provided on the under surface of the convex part of second holder 25b forming the periphery of negative pressure chamber 28a.

In addition, second skin detecting sensor 28f is provided on the upper surface of the concave part of second holder 25b forming negative pressure chamber 28a. Here, first skin detecting sensor 28b and second skin detecting sensor 28f may be one or more points, or may have a ring shape to surround the bottom of hole 22m.

First holder 25a has connectors 49 (49a to 49f). These connectors 49 (49a to 49f) are provided corresponding to connection electrodes 41a to 45a and identification electrode 47a (see FIG. 6(B)) provided on blood sensor 23. Signals of connection electrodes 41a to 45a and identification electrode 47a are guided to electrical circuit section 27 through connectors 49.

Laser light 26h emitted from laser emitting device 26 passes straight through hole 25g of first holder 25a, opening 34 of sensor blood 23 and hole 25m of second holder 25b, and punctures skin 9. When skin 10 is punctured, blood 11 exudes from skin 10, and forms blood droplet 11a.

Next, an operation in a case where laser emitting device 26 is used as a puncturing means will be described.

When skin 9 touches blood test apparatus 21, a signal is output from first skin detecting sensor 28b. High voltage generating circuit 27h activates according to the signal output from first skin detecting sensor 28 to allow the laser emitting device 26 to emit laser light 26h. This laser light 26h passes straight through hole 25g of first holder 25a, opening 34 of blood sensor 23 and hole 25m of second holder 25b to puncture skin 9. When the skin 9 is punctured, blood 10 exudes from skin 9 and blood droplet 10a is stored in opening 34. This blood droplet 10a is guided through supply path 35 by capillary action, is taken into detecting section 37 (see FIG. 5) and reacts with reagent 40. The signal reacted with reagent 40 is measured by electrical circuit section 27 through connectors 49.

FIG. 14 is a block diagram of electrical circuit section 27-1 and its periphery constituted by only puncturing apparatus 20.

In FIG. 14, electrical circuit section 27-1 has control section 27g. The output of control section 27g is connected to high voltage generating circuit 27h to which laser emitting device 26 is connected, and negative pressure means 28. In addition, cover detecting sensor 22e that detects opening and closing of cover 22b, charge completion detecting section 27m that detects that high voltage generating circuit 27h has been charged, first skin detecting sensor 28b, second skin detecting sensor 28f, current change detecting section 28g that detects change in the current supplied to negative pressure means 28 and timer 27k are connected to the input of control section 27g.

FIG. 15 is a block diagram of electrical circuit section 27-2 and its periphery of blood test apparatus 21. Here, the same components as in FIG. 14 will be assigned the same reference numerals.

In FIG. 15, connection electrodes 41a to 45a and identification electrode 47a (see FIG. 8) are connected to switching circuit 27a through connectors 49a to 49e. The output of this switching circuit 27a is connected to the input of current/voltage convertor 27b. Then, the output of current/voltage convertor 27b is input to computing section 27d through analog/digital convertor 27c (hereinafter referred to as “A/D convertor”). The output of computing section 27d is input to display section 50 made of liquid crystal and control section 27g. Reference voltage source 27f is connected to switching circuit 27a. Here, reference voltage source 27f may be a ground potential.

Control section 27g controls operation of the entire test apparatus according to the present invention. The output of control section 27g is input to high voltage generating circuit 27h connected to laser emitting device 26, a control terminal of switching circuit 27a, computing section 27d, communicating section 27e, negative pressure means 28 and conveying means 30. In addition, cover detecting sensor 22e that detects opening and closing of the cover 22b, charge completion detecting section 27m that detects that high voltage generating circuit 27h has been charged, first skin detecting sensor 28b, second skin detecting sensor 28f, computing section 27d, current change detecting section 28g that detects change in the current supplied to negative pressure means 28, timer 27k and connector 49f connected to identification electrode 47a are connected to the input of control section 27g.

Next, the operation of electrical circuit section 27-2 will be described.

First, when first skin detecting sensor 28b detects skin 9, negative pressure means 28 is activated to create a negative pressure in negative pressure chamber 28a. In addition, high voltage generating circuit 27h starts charging a capacitor. Next, laser emitting device 26 punctures skin 9 at one of: the time at which second skin detecting section detects that skin 9 swells; the time at which timer 27k measures a predetermined passage of time; and the time at which current change detecting section 28g detects change in the current supplied to negative pressure means 28, and also the time at which charge completion detecting section 27m detects that the capacitor is sufficiently charged via high voltage generating circuit 27h (third example of ready for puncturing).

Then, the property of blood 10 exuded by puncturing is measured. In the measuring operation, detection electrodes 41 (see FIG. 6) are connected to current/voltage convertor 27b by switching switching circuit 27a. In addition, detection electrode 42 to be electrode for detecting an inflow of blood 10 is connected to reference voltage source 27f. Then, a constant current is applied between the detection electrode 41 and the detection electrode 42. In this state, a current flows between detection electrodes 41 and 42 if blood 10 flows in. This current is converted into a voltage by current/voltage convertor 27b, and the voltage value is converted into a digital value by A/D convertor 27c. Then, the digital value is output to computing section 27d. Computing section 27d detects that blood 10 has sufficiently flown in, based on the digital value, and outputs this fact to the control section 27g.

Here, at this time, the operation of pump 28 is turned off by a command from control section 27g.

Next, glucose being a component of blood will be measured. For measuring the glucose level, first, switching circuit 27a is switched by a command from control section 27g, and detection electrode 41 to be a working electrode for measuring glucose level is connected to current/voltage convertor 27b. In addition, detection electrode 43 to be a counter electrode for measuring the glucose level is connected to reference voltage source 27f.

Here, for example, while the glucose in blood and its oxidation-reduction enzyme react for a given period of time, current/voltage convertor 27b and reference voltage source 27f are turned off. Then, after the certain period of time (1 to 10 seconds) passes, a certain voltage (0.2 to 0.5 V) is applied between detection electrodes 41 and 43 by a command from control section 27g. By this means, a current flows between detection electrodes 41 and 43. This current is converted into a voltage by current/voltage convertor 27b, and the voltage value is converted into a digital value by A/D convertor 27c and output to computing section 27d. Computing section 27d converts this digital value to a glucose level.

Next, after the glucose level is measured, Hct value will be measured. The Hct value will be measured as follows. Firstly, switching circuit 27a is switched by a command from control section 27g. Then, detection electrode 45 to be a working electrode for measuring the Hct value is connected to current/voltage convertor 27b. In addition, detection electrode 41 to be a counter electrode for measuring the Hct value is connected to reference voltage source 27f.

Next, a certain voltage (2V to 3V) is applied between detection electrodes 45 and 41 from current/voltage converter 27b and reference voltage source 27f, by a command from control section 27g. The current flowing between detection electrodes 45 and 41 is converted into a voltage by current/voltage convertor 27b, and the voltage value is converted into a digital value by A/D converter 27c and output to computing section 27d. Computing section 27d converts this digital value to Hct value.

By using the Hct value and glucose content resulting from measurement and referring to a calibration curve or calibration curve table determined in advance, glucose content is corrected by the Hct value and the correction result is displayed on display section 55. The calibration curve or calibration curve table is determined by identifying section 47 in sensor 23. In addition, the result of correction using the calibration curve or calibration curve table is transmitted from communicating section 27e to an injection device for injecting insulin. Although a radio wave may be used for this transmission, transmission is preferably performed by optical communication that does not interfere with medical equipment.

When the dose of insulin to administer is automatically set by transmitting corrected measurement data from transmitting section 27e in this way, setting the dose of insulin to be administered by the patient is not required, which eliminates botheration with setting. Moreover, since the dose of insulin can be set in the injection device without human work, setting error can be prevented.

Although an example of glucose measurement has been described above, the blood test apparatus is applicable to measurement of blood components other than glucose such as lactate acid or cholesterol levels by changing reagent 40 of sensor 23.

Next, operation steps for a case where only puncturing apparatus 20 (not shown) will be explained.

Puncturing apparatus 20 has a configuration including laser emitting device 26, negative pressure means 28, cover 22b and second holder 25b among the components of the above-described blood test apparatus 21.

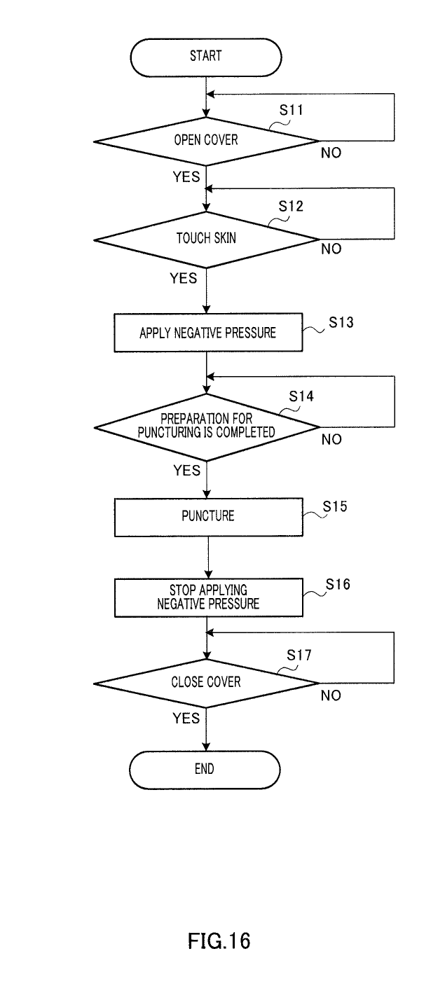

FIG. 16 is a flowchart showing a puncturing method for puncturing apparatus 20. Control section 27g may include a microprocessor, for example. The flow is repeatedly performed by this microprocessor at a predetermined timing.

First, in step S11, cover detecting sensor 22e detects opening of cover 22b of puncturing apparatus 20. Here, when cover 22b of puncturing apparatus 20 does not open, the step does not go forward. A detection signal from cover detecting sensor 22e is input to control section 27g. Receiving this detection signal, the control section 27g turns on the power supply of puncturing apparatus 20 and displays an indication to prompt holding section 25 to contact skin 9 on display section 50 (see FIG. 15). The step moves to step S12 following the displaying.

In step S12, first skin detecting sensor 28b detects whether the patient attaches puncturing apparatus 20 to his/her skin 9 according to a command from display section 50. If skin 9 does not touch puncturing apparatus 20, first skin detecting sensor 28b waits until skin 9 touches puncturing apparatus 20. When first skin detecting sensor 28b detects that skin 9 touches puncturing apparatus 20, the step moves to step S13.

In step S13, control section 27g makes negative pressure means 28 operate to apply a negative pressure to inside negative pressure chamber 28a provided in holding section 25. In addition, control section 27g makes high voltage generating section 27h start charging.

Skin 9 swells by applying the negative pressure as shown in FIG. 13.

In step S14, control section 27g determines whether preparation for puncturing is completed. Specifically, control section 27g determines that preparation for puncturing is completed when the following conditions are satisfied. That is, control section 27g determines that preparation for puncturing is completed when a first information and a second information are satisfied, where the first information is one of output information from second skin detecting sensor 28f that detects the swelled skin 9; output information from current change detecting section 28g that detects change in the current of negative pressure means 28 resulting from the operation of negative pressure means 28; and information that timer 27k measures a predetermined time passage (1 to 10 seconds), and the second information is based on the output of charge completion detecting section 27m that detects that charging by high voltage generating circuit 27h is completed (1 to 10 seconds). On the other hand, if the first information and the second information are not satisfied, control section 27g determines that preparation for puncturing is not completed, and waits until the preparation for puncturing is completed. When the preparation for puncturing is completed, the step moves to step S15 (puncturing step).

In step S15, control section 27g punctures skin 9 using laser emitting device 26. By puncturing skin 9, blood 10 exuding from skin 9 can be tested by another measuring device. After skin 9 is punctured, the step moves to step S16.

In step S16, control section 27g stops negative pressure means 28 after a predetermined time passes since the puncturing has been performed. After negative pressure means 28 is stopped, the step moves to step S17.

In step S17, cover detecting sensor 22e detects the closed state of cover 22b of puncturing apparatus 20. Receiving the detection signal, control section 27g turns off the power supply of puncturing apparatus 20 and ends the flow. Here, when the closed state of cover 22b of puncturing apparatus 20 is not detected, the step does not go forward.

Next, a test method of blood test apparatus 21 will be described.

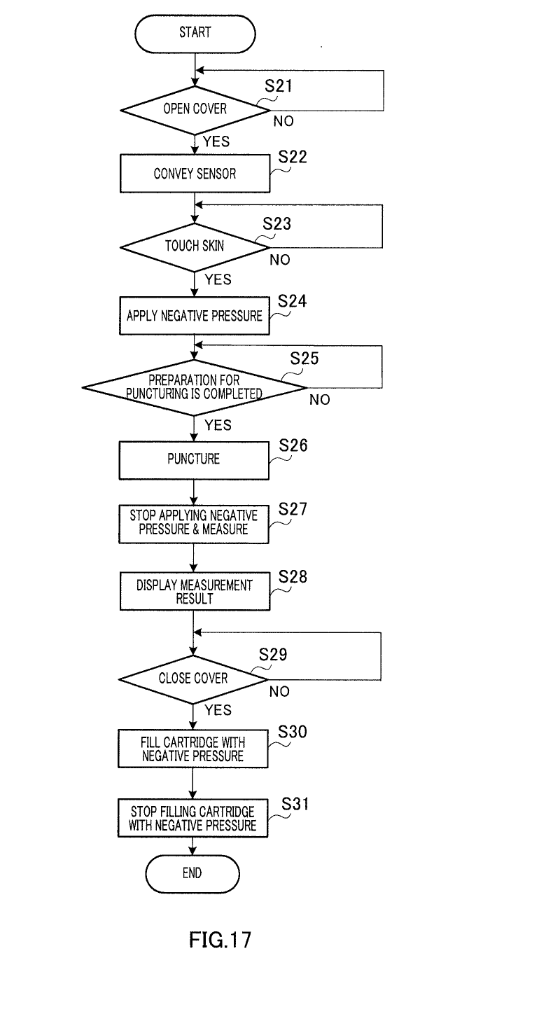

FIG. 17 is a flowchart showing a test method of blood test apparatus 21.

First, in step S21, cover 22b is opened. In conjunction with this, shutter 24n of sensor outlet 24a provided in cartridge 24 is opened. At this time, cover detecting sensor 22e detects the opened state of cover 22b of puncturing apparatus 22b. A detection signal from cover detecting sensor 22e is input to control section 27g. Receiving this detection signal, control section 27g turns on the power supply of puncturing apparatus 20. Then the step moves to step S22. Here, when the opening state of cover 22b of puncturing apparatus 20 is not detected, the step does nor go forward.

In step S22, control section 27g activates conveying means 30 to make first holder 25a and second holder 25b sandwich and fix blood sensor 23. It is possible to check that sensor 23 has been conveyed by detecting electric conduction between connection electrode 43a and identification electrode 47a of sensor 23 fixed to the holding section. Based on this detection of electric conduction, control section 27g displays an indication to prompt holding section to contact skin 9 on the display section 50 (see FIG. 15). Following the displaying, the step moves to step S23.

In step S23, the patient touches puncturing apparatus 20 with skin 9 according to a command from display section 50. First skin detecting sensor 28b detects the touch. When skin detecting sensor 28b detects the touch with skin 9, the step moves to step S24.

In step S24, control section 27g activates negative pressure means 28 to apply a negative pressure to inside negative pressure chamber 28a provided in holding section 25. In addition, control section 27g makes high voltage generating circuit 27h start charging, and the step moves to step S25.

Here, skin 9 swells as shown in FIG. 13 by applying the negative pressure.

In step S25, control section 27g determines whether preparation for puncturing is completed. Specifically, control section 27g determines that preparation for puncturing is completed when the following respective conditions are satisfied. That is, control section 27g determines that preparation for puncturing is completed when a first information and a second information are satisfied, where the first information is one of output information from second skin detecting sensor 28f that detects the swelled skin 9; output information from current change detecting section 28g that detects change in the current of negative pressure means 28 along with the operation of negative pressure means 28; and information that timer 27k measures a predetermined time passage (1 to 10 seconds), and the second information is based on the output of charge completion detecting section 27m that detects that charging by high voltage generating circuit 27h is completed (1 to 10 seconds). Meanwhile, if the first information and the second information are not satisfied, control section 27g determines that preparation for puncturing is not completed, and waits until the preparation for puncturing is completed.
When the preparation for puncturing is completed, the step moves to step S26.

In step S26, control section 27g automatically punctures skin 9 by laser emitting device 26. By puncturing skin 9, blood 10 exuding from skin 9 can be tested by another measuring device. After skin 9 is punctured, the step moves to step S27.

In step 27, control section 27g stops negative pressure means 28 and performs measurement. The measurement will be performed as follows. Exuded blood 10 by puncturing skin 9 is taken into opening 34 of blood sensor 23 (see FIG. 13). Blood 10 taken into opening 34 is guided to detecting section 37 by capillary action through supply path 35, and the blood sugar level is measured, then the step moves to step S28. Here, negative pressure means 28 may be turned off at the time the measurement of the blood sugar level is completed or blood 10 reaches detection electrode 42.

In step S28, the result of the measured blood sugar level is displayed on display section 55, and the step moves to step S29. Here, the measurement result of the blood sugar level may be automatically transmitted from communicating section 27e to another device such as an injection device.

In step S29, cover detecting sensor 22e detects the closed state of cover 22b of puncturing apparatus 20. Shutter 24n of cartridge 24 moves in conjunction with closing cover 22b by the patient to close sensor outlet 24a. Cover detecting sensor 22e detects the closed state of cover 22b and notifies the control section 27g, then the step moves to step S30.

In step S30, when cover detecting sensor 22e detects the closed state of cover 22b, control section 25g applies a negative pressure to inside cartridge 24 for a predetermined time, and the step moves to step S31. Here, the reason why the negative pressure is applied to inside cartridge 24 is to remove the dampness in cartridge 24 to alleviate deterioration of blood sensor 23 due to the dampness.

In step S31, after applying a negative pressure to inside the cartridge 24 for a predetermined time, control section 27g stops applying the negative pressure and turns off the power supply of puncturing apparatus 20 to end this flow.

As described above, the only things to do for the patient are to open cover 22b in step S21 and to attach skin 9 to holding section 25, and all the subsequent operations of the blood test are automatically performed at appropriate timings. Therefore, the burden of operation is removed.

Moreover, since puncturing is performed through opening 34 (see FIG. 13), exuded blood 10 can be all utilized efficiently because all exuded blood 10 is accumulated in opening 34, thereby minimizing the burden on the patient and also improving the reliability significantly.

### Embodiment 2

In embodiment 1, all operations for a blood test can be automatically performed at appropriate timings by only touching holding section 25 with skin 9.

In embodiment 2, timings for puncturing will be described in detail.

For puncturing apparatus 20 and blood test apparatus 21 according to embodiment 2, “preparation for puncturing” will be defined as follows.

(1) Preparation for Puncturing

The preparation for puncturing refers to a process performed before puncturing in order to allow puncturing apparatus 20 and blood test apparatus 21 to surely puncture.

Specifically, the flowing processes are involved.

a. charging a capacitor with the voltage required for puncturing with laser light.

b. swelling the surface of skin up to the reference position for determining the puncture depth.

(2) Start Preparation for Puncturing

“Start preparation for puncturing” refers that blood test apparatus 21 detects that skin 9 touches the bottom of second sensor holder 25b to start the preparation for puncturing.

(3) Preparation for Puncturing is Completed

“Preparation for puncturing is completed ” refers to a condition where the capacitor is charged with the voltage required for puncturing with laser light and the surface of skin is swelled up to the reference position for determining the puncture depth.

Here, for laser puncturing, the reference position for determining the puncture depth is the focal position of laser light, and for needle puncturing, the reference position for determining the puncture depth is the depth of a puncture needle.

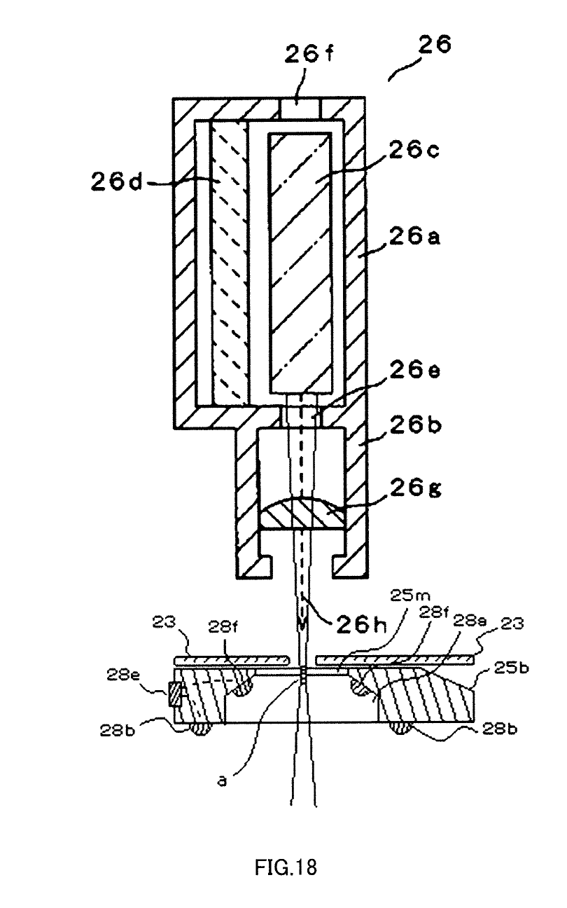

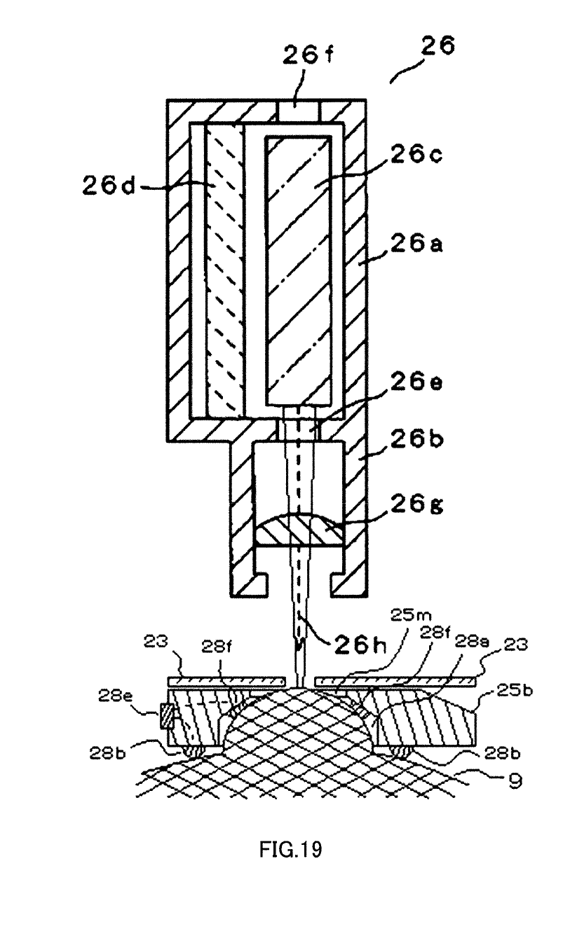

FIG. 18 and FIG. 19 are drawings explaining the reference position for determining the puncture depth of a puncturing apparatus according to embodiment 2 of the present invention. Here, the same components as in FIG. 9 and FIG. 13 will be assigned the same reference numerals and explanation for overlapping parts will not be repeated.

In FIG. 18, hole 25m of second holder 25b faces a laser light emitting outlet of cylindrical body 26b of laser emitting device 26, and blood sensor 23 is placed on second holder 25b. Blood sensor 23 is the undermost blood sensor separated one-by-one from blood sensors 23 stacked and stored in cartridge 24, and conveyed onto second holder 25b using conveying unit 30.

Hole 25m in communication with hole 25g formed on first holder 25a is provided on second holder 25b. In addition, negative pressure chamber 28a opening downward is coupled with hole 25m. Moreover, first skin detecting sensor 28b is provided adjacent to negative pressure chamber 28a, and second skin detecting sensor 28f that detects the swelled skin when a negative pressure is applied to negative pressure chamber 28a or when the skin is pressed is provided on the upper side of negative pressure chamber 28a.

Laser light 26h of laser emitting device 26 is set so as to focus on a point near the surface of the skin of the patient. In this case, point a., slightly below hole 25m formed on second holder 25b in FIG. 18, is the focal point of the laser light, that is the reference position for determining the puncture depth.

Laser light 26h emitted from laser emitting device 26 passes through condenser lens 26g and is converged to focus on the position a. as shown in FIG. 18.

As shown in FIG. 19, skin 9 swells when the negative pressure is applied to negative pressure chamber 28a or the skin is pressed. Skin 9 touches the first detecting sensor 28b provided on second holder 25b, and first skin detecting sensor 28b detects the skin, Furthermore, swelled skin 9 also touches second skin detecting sensor provided on upper surface of negative pressure chamber 28a, and second skin detecting sensor 28f detects the skin. Here, although skin 9 swells by applying a negative pressure to negative pressure chamber 28a, the present invention is limited to this, and the skin also swells by being strongly pressed against second holder 25b.

Laser light 26h passes through condenser lens 26g, is converged and focuses on a position adjacent to the surface of swelled skin 9. The depth of the focal point in puncturing is preferably 0.6 mm to 1.5 mm from the surface of skin 9. Here, the depth is 0.8 mm in the present embodiment.

The surface of the swelled skin 9 is positioned at the focal position of laser light 26h, so that puncturing can be surely performed.

The relationship between swelling of skin and the preparation for puncturing will be described later.

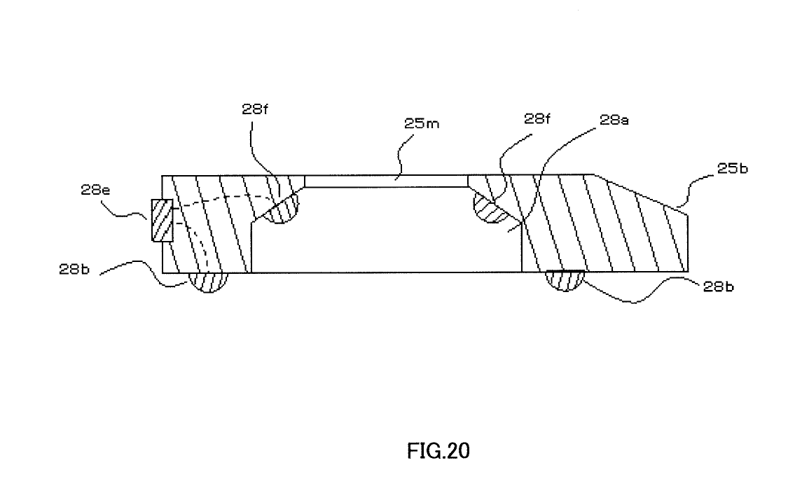

FIG. 20 to FIG. 24 are drawings explaining a method of detecting swelled skin. Here, the same components as in FIG. 18 and FIG. 19 will be assigned the same reference numerals

FIG. 20 is a cross sectional view showing the second holder 25b from the side. First skin detecting sensor 28b is provided adjacent to negative pressure chamber 28a of second holder 25b, and second skin detecting sensor 28f is provided on the upper side of negative pressure chamber 28a. First skin detecting sensor 28b and second skin detecting sensor 28f are connected to electrical circuit section 27 (see FIG. 4) through connector 28e.

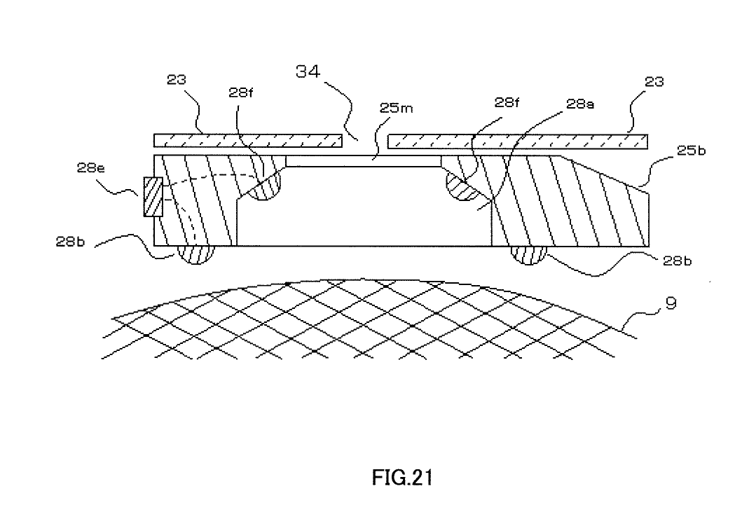

FIG. 21 shows a state where skin 9 does not touch the bottom end of second holder 25b and first skin detecting sensor 28b does not detect skin 9. As shown in FIG. 21, blood sensor 23 is placed on second holder 25b. This blood sensor 23 is the undermost blood sensor separated one-by-one from the blood sensors 23 stacked and stored in cartridge 24 and conveyed onto second holder 25b using conveying unit 30. Blood sensor 23 is provided with opening 34 allowing the puncturing part of the puncturing means to pass through. In addition, opening 34 has a function for storing therein the exuded blood. The puncturing means is laser light 26h when laser emitting device 26 is used, and it is a puncture needle when a needle puncture device is used.

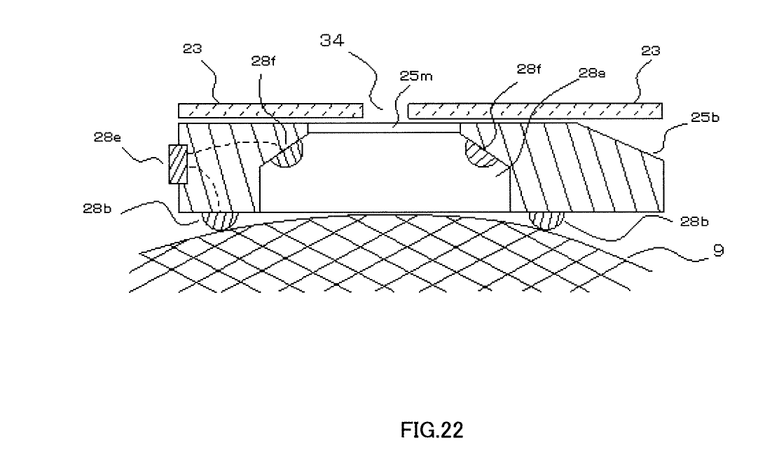

FIG. 22 is a drawing showing a state where skin 9 touches the lower end of second holder 25b and first skin detecting sensor 28b detects skin 9.

This state causes preparation for puncturing to start.

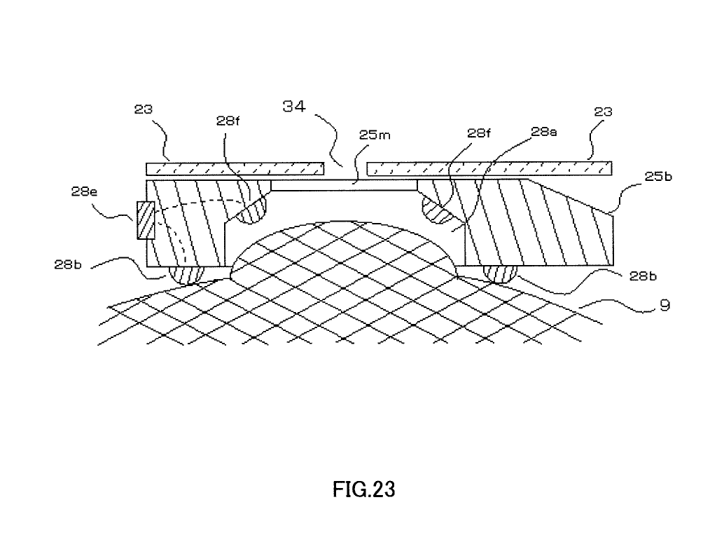

FIG. 23 is a drawing showing a state where a skin swelling operation is started by the negative pressure from negative pressure chamber 28a or by pressing skin 9, and then the skin begins to swell. Skin 9 has been in contact with first skin detecting sensor 28b, but has not been in contact with second skin detecting sensor 28f provided on the upper surface of negative pressure chamber 28a yet.

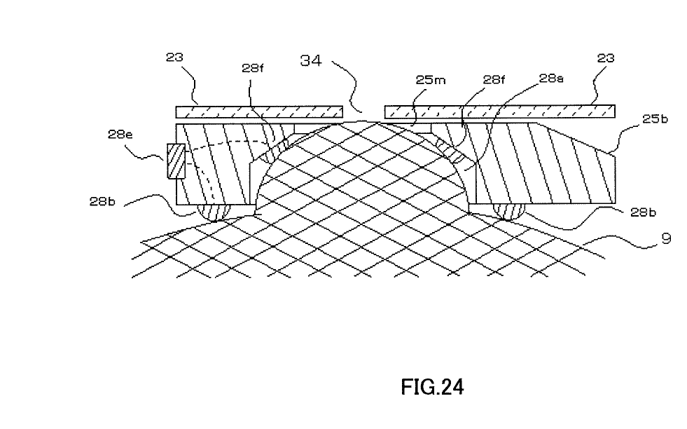

FIG. 24 is a drawing showing a state where the skin is further swelled by the negative pressure from negative pressure chamber 28a or by continuously pressing skin 9 and touches second skin detecting sensor 28f provided on the upper surface of negative pressure chamber 28a, so that second skin detecting sensor 28f detects the skin.

By this means, the preparation for puncturing is completed, and puncturing can be performed all the time.

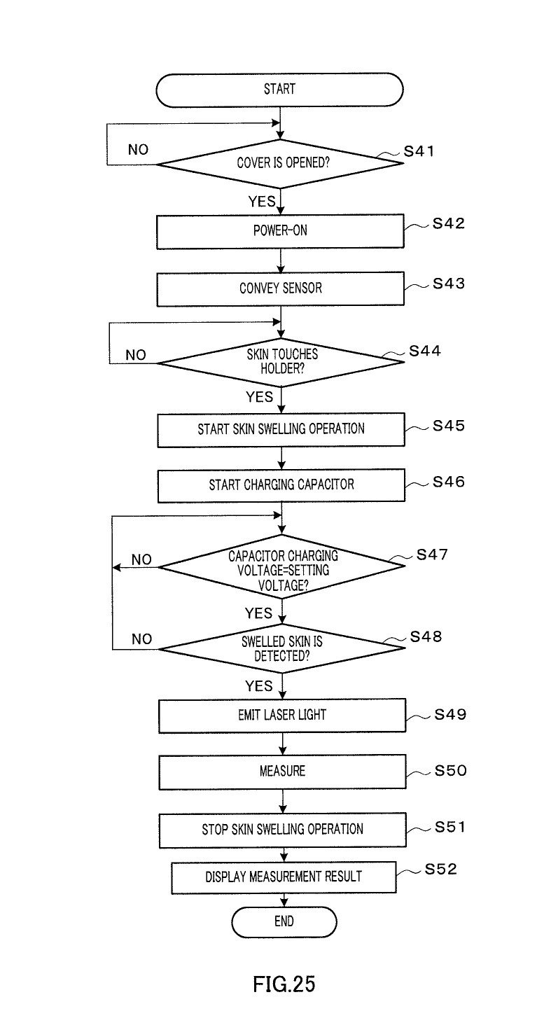

FIG. 25 shows a flow chart showing a puncturing-and-measuring method for puncturing apparatus 20.

First, in step S41, cover detecting sensor 22e detects an open state of cover 22b of puncturing apparatus 20. A detection signal by cover detecting sensor 22e is input to control section 27g and the step moves to step S42. Here, if cover 22b of puncturing apparatus 20 is not opened, the step does not go forward.

When detecting the opening state of cover 22b of puncturing apparatus 20, control section 27g turns on the power supply of puncturing apparatus 20 in step S42. In addition, when cover 22b of blood test apparatus 21 is opened, shutter 24n of sensor outlet 24a provided on cartridge 24 opens in conjunction with the opening of cover 22b, and then the step moves to step S43.

In step S43, control section 27g receives a signal from cover detecting sensor 22e and activates conveying means 30. Slider 24k constituting conveying means 30 is moved toward sensor outlet 24a, so that only one blood sensor 23, which is the undermost blood sensor 23 is separated from the blood sensors 23 stacked and stored, ejected and moved from sensor outlet 24a, and conveyed to holding section 25 (see FIG. 8). It is possible to check that sensor 23 has been conveyed by detecting electric conduction between connection electrode 43a and identification electrode 47a of sensor 23. Then, slider plate 24k constituting conveying means 30 returns to a standby state by a repulsive force of spring 24p, and it is possible to prepare for conveying the next sensor. The conveyed blood sensor 23 is sandwiched and fixed between first holder 25a and second holder 25b of holding section 25, and the step moves to step S44.

In step S44, when the skin touches the bottom end of second holder 25b, control section 27g receives a detection signal from first skin detecting sensor 28b, and the step moves to step S45 (see FIG. 22).

In step S45, control section 27g makes negative pressure means 28 apply a negative pressure to inside negative pressure chamber 28a provided in holding section 25 to start a skin swelling operation, and the step moves to step S46.

In step S46, control section 27g causes high voltage generating circuit 27h to start charging the capacitor of laser emitting device 26, and the step moves to step S47. Here, the processes of step S45 and S46 correspond to “preparation for puncturing”

In step S47, control section 27g determines whether the capacitor is charged up to the predetermined setting voltage, based on a charge completion detecting signal from charge completion detecting section 27m provided on high voltage generating circuit 27h, and the step moves to step S48. Here, this setting voltage is a capacitor charging voltage for which high voltage generating circuit 27h makes excitation light source 26d emit laser light.

In step S48, control section 27g detects that the skin has touched second skin detecting sensor 28f provided on the upper surface of negative pressure chamber 28a with respect to swelled skin required for puncturing-and-measuring, and the step moves to step S49.

Here, when the capacitor is not charged up to the setting voltage in step S47, or when the swelled skin required for puncturing is not detected in step S48, the step returns to the above-described step S47, and waits until the capacitor is charged with the setting voltage and also the skin swells enough to perform puncturing.

In step S49, laser light is emitted. Specifically, control section 27g outputs a laser emitting signal to laser emitting device 26, and laser emitting device 29 receives this laser emitting signal to activate high voltage generating circuit 27h (see FIG. 15). At this time, since the capacitor has been already charged with enough voltage to emit the laser light, high voltage generating circuit 27h activates and laser emitting device 26 emits laser light 26h, and the step moves to step S50. Here, laser light 26h passes straight through hole 25g, opening 34 and hole 25m to puncture skin 9.

In step S50, electrical circuit section 27 performs various measurements, based on blood 10 sampled by puncturing. When skin 9 is punctured, blood 10 exudes from this skin 9 and forms blood droplet 10a in opening 34. Blood droplet 10a is guided to detecting section 37 by capillary action and reacts with reagent 40. As a result of the reaction with reagent 40, an obtained signal is measured by electrical circuit section 27 through connector 49, and the step moves to step S51. Here, the blood test apparatus is applicable to measurement of blood components other than glucose such as lactate acid or cholesterol levels by changing reagent 40.

In step S51, control section 27g stops negative pressure means 28 to stop applying a negative pressure to cartridge 24 and swelling the skin, and the step moves to step S52. Here, the atmosphere may be introduced at the same time as negative pressure means 28 is stopped.

In step S52, control section 27g displays the result of the measured blood sugar level and so forth on display section 55, and this flow ends. Here, this measurement result may be transmitted from communicating section 27e to another device such as an injection device.

### Embodiment 3

In embodiment 2, the best mode of the puncturing-and-measuring method for puncturing apparatus 20 has been illustrated. According to embodiments 1 and 2, the automatic puncturing at an appropriate puncturing timing can be achieved only by attaching puncturing apparatus 20 to the skin. However, actually there is a case where puncturing, or puncturing-and-measuring is not appropriately performed by various factors. In embodiment 3, a puncturing-and-measuring method when a failure occurs will be described.

A failure will occur when (1) preparation for puncturing is not completed and (2) puncturing-and-measuring is not successful. Each occurrence of a failure is informed to the user through an error information when the timer measures a predetermined time passage.

First, the instance (1) preparation for puncturing is not completed will be described.

As an example for an instance where preparation for puncturing is not completed, it is assumed that the user moves his/her finger (a part to be punctured).

FIG. 26 is a flowchart showing a puncturing-and-measuring method when a failure occurs because preparation for puncturing is not completed. Here, the steps of performing the same processing as in the flow of FIG. 25 will be assigned the same step numbers and the explanation will be omitted.

In step S44, when the skin touches the bottom end of second holder 25b, control section 27g starts counting by timer T1 in step S61. Then, in step S45, control section 27g starts a skin swelling operation, and in step S46, control section 27g starts charging a capacitor of laser emitting device 26, and the step moves to step S62.

In step S62, it is determined whether predetermined time t1 (e.g. 5 seconds to 10 seconds) has passed (T1≧t1) or not, based on the count of timer T1.

If predetermine time t1 has not passed (T1<t1), control section 27g determines that the finger (a part to be punctured) has not moved and the step moves to step S47 and subsequent steps, and then the processing the same as the puncturing-and-measuring flow in FIG. 25 will be performed.

If it is determined that predetermined time t1 has passed (T1≧t1), control section 27g determines that the finger (a part to be punctured) is moved, and the step moves to step S63. In this case, the skin does not touch second skin detecting sensor 28f after predetermined time t1 passes since the skin touches first skin detecting sensor 28b of the bottom end of second holder 25b. Usually, the skin swells by applying the negative pressure from negative pressure means 28 or by pressing the skin, so that the skin should touch second skin detecting sensor 28f within predetermined time t1. If it is not detected that the skin does not touch second skin detecting sensor 28f after predetermined time t1 passes, it is assumed that the user moves his/her finger (a part to be punctured). This is an example of an instance where preparation for puncturing is not completed.

In step S63, control section 27g stops charging the capacitor, and the step moves to step S64.

In step S64, control section 27g stops negative pressure means 28 to stop applying the negative pressure to the cartridge 24 and swelling the skin. Here, the atmosphere may be introduced at the same time as negative pressure means 28 is stopped.

In step S65, control section 27g issues error notification that preparation for puncturing has not been completed to the user, and this flow ends. The error notification is performed by indicating that the error occurs on the display section 50 (see FIG. 15), for example. In addition, the error notification may be performed by audio guidance using the speech synthesis LSI, making sound by a speaker, a LED display, or combination thereof.

Thus, when preparation for puncturing is not completed, it is assumed that the second skin detecting sensor can not detect that the skin swells because the user moves his/her skin after the preparation for puncturing starts. Therefore, if the preparation for puncturing is not completed after a predetermined time (5 seconds to 10 seconds) passes since the preparation for puncturing starts, error notification is issued to the user and thereby the user is prompted to perform measurement again.

Next, an instance where (2) puncturing-and-measuring is not successful will be described.

In this case, it is assumed that measurement is performed again immediately after measurement because the previous puncturing-and-measuring is not successful.

Since the temperature of the puncturing laser rises immediately after measurement, there is a case where puncturing-and-measuring cannot be surely performed if the subsequent puncturing is performed immediately after the previous puncturing is performed. In the present embodiment, therefore the apparatus does not perform to the step for starting the next preparation for puncturing even if the user operates the apparatus in order to perform measurement again immediately after measurement, and the apparatus notifies the user to wait because the subsequent measurement cannot be performed for a while.

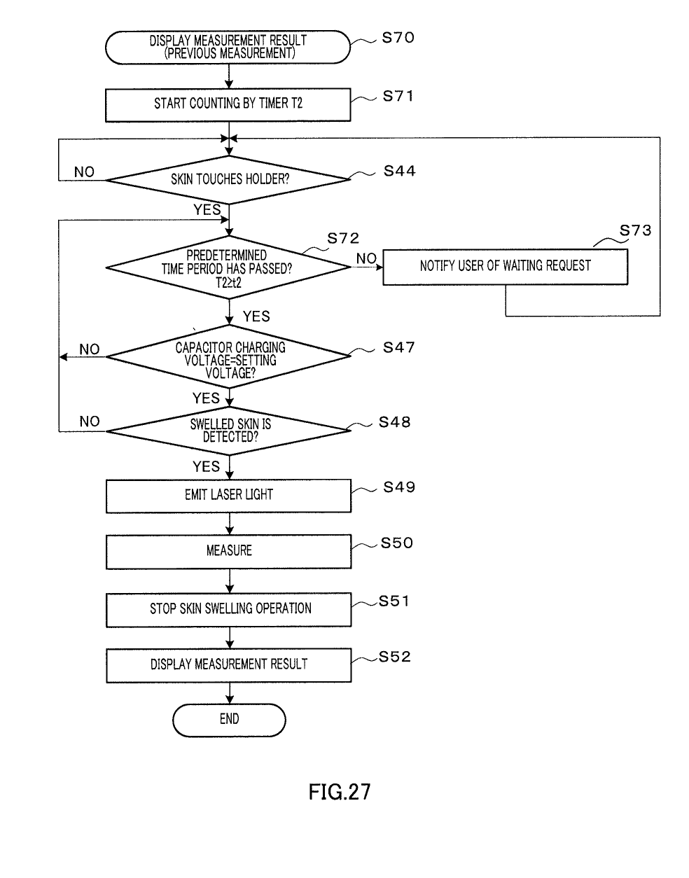

FIG. 27 is a flowchart showing a puncturing-and-measuring method when a failure occurs because of an unsuccessful puncturing-and-measuring. Here, the steps performing the same processing as in the flow of FIG. 25 will be assigned the same step numbers and the explanation will be simplified.

In step S70, the result of the previous measurement is displayed on display section 55.

In step S71, control section 27g starts counting by timer T2. Then, the step moves to step S44.

When the skin touches the bottom end of second holder 25b in the above-described step S44, control section 27g determines whether predetermined time t2 (e.g. 2 seconds to 3 seconds) has passed (T2≧t2) or not, based on the count of timer T2.

When predetermined time t2 passes (T2≧t2), control section 27g determines that the preparation for puncturing is completed, goes to step S47 and performs the same processing as the puncturing-and-measuring flow in FIG. 25.

In the above-described step S72, if predetermined time t2 has not passed (T2<t2), control section 27g notifies the user to wait in step S73 and returns to the above-described step S44. The wait request notification may be performed by indicating that the error occurs on the display section 50 (see FIG. 15), for example.

Thus, if remeasurement is performed immediately after the previous measurement is performed because puncturing-and-measuring is unsuccessful, since the temperature of the puncturing laser rises immediately after measurement, there is a case where puncturing-and-measuring cannot be surely performed. At a time like this therefore the apparatus does not perform to the step for starting the next preparation for puncturing even if the user operates the apparatus in order to perform measurement again (i.e. touching the holder with the finger) immediately after measurement, and the apparatus notifies the user to wait because the subsequent measurement cannot be performed for a while. In the present embodiment, remeasurement cannot be performed using timer T2 until a predetermined time period (2 to 3 minutes) passes after measurement.

The puncturing-and-measuring method when a failure occurs as shown in FIG. 26 and the puncturing-and-measuring method when a failure occurs as shown in FIG. 27 may be combined.

Although preferred embodiments of the present invention have been illustrated, the scope of the preset invention is not to limited to this.

For example, although laser emitting device 26 is adopted as a puncturing means in each of the above-described embodiments, the present invention is not to limited to this, and a puncturing device that punctures with a puncture needle as a puncturing means may be adopted.

In addition, although second skin detecting sensor 28f detects swelled skin in each of the above-described embodiment, a current detecting section that detects change in the current in the negative pressure means may be used instead.

Moreover, although swelling of skin is created by the negative pressure applied from negative pressure section 28, the swelling may be created by pressing the skin, not only by the negative pressure. In addition, the name “negative pressure chamber 25” is only a convenient name for using a negative pressure, and may be an “opening” (“concave part” “space section”, etc) and so forth.

Furthermore, although the names “puncturing apparatus”, “blood test apparatus” and “puncturing method” are used in the present embodiment for convenience of explanation, it goes without saying that the name of the apparatus may be a “test apparatus”, and the name of the method may be a “method for controlling a puncturing apparatus” and so forth.

Moreover, for each component constituting the puncturing apparatus and the blood test apparatus, such as a cartridge, the kind, the number and the connection method thereof may be selected arbitrarily.

The above-described puncturing method can be provided by a program for functioning the puncturing method. The program is stored on a computer-readable recording medium.

The present invention claims priority based on Japanese Patent Application No. 2007-186641, filed on Jul. 18, 2007. The disclosure including the specification and drawings as filed, is incorporated herein by reference in its entirety.

### INDUSTRIAL APPLICABILITY

The present invention is applicable to a blood test apparatus that punctures skin by a puncturing means, for example, a laser emitting device, samples blood exuding from the skin and analyzes components of the blood.

1. A puncturing apparatus comprising:
a housing;
a puncturing section that is provided in the housing;
a first skin detecting section that detects that skin touches a predetermined puncturing position;
a second skin detecting section that detects swelled skin after the first detecting section detects contact with the skin;
a control section that allows the puncturing section to puncture the swelled skin when the second skin detecting section detects the swelled skin.

2. The puncturing apparatus according to claim 1, further comprising a negative pressure section that applies a negative pressure to inside the housing to swell the skin.

3. The puncturing apparatus according to claim 2, wherein the second skin detecting section has a current detecting section that detects change in a current of the negative pressure section, and detects swelled skin based on the change in the current detected by the current detecting section.

4. The puncturing apparatus according to claim 1, wherein the puncturing section is a laser emitting device that punctures the skin with laser light out of touch.

5. The puncturing apparatus according to claim 1, wherein the puncturing section is a needle puncture device that punctures the skin with a puncture needle.

6. The puncturing apparatus according to claim 4, further comprising:
a capacitor that accumulates a charge for emitting the laser light by the laser emitting device; and
a charge completion detecting section that detects that charging on the capacitor is completed,
wherein the control section detects swelled skin by the second skin detecting section and allows the puncturing section to puncture the skin when the charge completion section detects that the charging is completed.

7. The puncturing apparatus according to claim 4, further comprising:
a capacitor that accumulates a charge for emitting the laser light by the laser emitting device; and
a charge completion detecting section that detects that charging on the capacitor is completed,
wherein the control section starts charging on the capacitor when the first skin detecting section detects the skin.

8. The puncturing apparatus according to claim 6, further comprising a first timer that measures a predetermined time period after the first skin detecting section detects contact with the skin,
wherein the control section stops charging the capacitor when the first timer measures the predetermined time period.

9. The puncturing apparatus according to claim 1, further comprising a second timer that measures a predetermined time period after a previous puncturing is terminated,
wherein the control section prohibits the puncturing section from puncturing until the second timer measures the predetermined time period.

10. The puncturing apparatus according to claim 8, further comprising a notification section that notifies of an error when charging on the capacitor is stopped.

11. The puncturing apparatus according to claim 9, further comprising a notification section that notifies of a waiting request when the puncturing section is prohibited from puncturing.

12. A blood test apparatus comprising a puncturing section that punctures skin though a blood sensor to test blood exuding on the blood sensor by puncturing, wherein a puncturing apparatus according to claim 1 is used as the puncturing section.

13. The blood test apparatus according to claim 12, further comprising:
a cartridge that stacks and stores blood sensors; and
a conveying section that conveys the blood sensors stacked and stored in the cartridge to a predetermined puncturing position one-by-one.

14. A puncturing method comprising:
detecting that skin touches a predetermined puncturing position of a housing;
detecting swelled skin after detecting contact with the skin; and
puncturing the swelled skin when detecting the swelled skin.

15. A puncturing method comprising:
detecting that skin touches a predetermined puncturing position of a housing;
applying a negative pressure to inside the housing to swell the skin after detecting contact with the skin;
detecting swelled skin; and
puncturing the swelled skin when detecting the swelled skin.

16. A puncturing method comprising:
detecting that skin touches a predetermined puncturing position of a housing;
applying a negative pressure to inside the housing to swell the skin after detecting contact with the skin;
starting charging a capacitor for emitting laser light;
detecting that charging on the capacitor is completed;
detecting swelled skin; and
puncturing the swelled skin with the laser light when detecting the swelled skin and detecting that the charging is completed.

17. The puncturing method according to claim 14 further comprising testing blood exuding on a blood sensor by puncturing.

18. The puncturing method according to claim 17 further comprising conveying the blood sensor to be measured, from a cartridge that stacks and stores blood sensors to a predetermined puncturing position one-by-one.
